(19) United States
(12) Patent Application Publication
(10) Pub. No.: US 2004/0234521 A1
(43) Pub. Date: Nov. 25, 2004

(54) Polyclonal populations of bispecific molecules and methods of production and uses thereof

(75) Inventor: Jeff Himawan, San Jose, CA (US)

Correspondence Address: JONES DAY, 222 EAST 41ST ST, NEW YORK, NY, 10017, US

(73) Assignee: Elusys Therapeutics, Inc

(21) Appl. No.: 10/472,051

(22) PCT Filed: Mar. 14, 2002
(86) PCT No.: PCT/US02/07950

Related U.S. Application Data
(60) Provisional application No. 60/276,200, filed on Mar. 15, 2001.

(51) Int. Cl. A61K 39/395; C07K 16/28

(52) U.S. Cl. 424/144.1; 530/388.22

(57) ABSTRACT

The invention relates to a polyclonal population of bispecific molecules which comprises a plurality of different bispecific molecules, each comprising a first antigen recognition portion that binds a C3b-like receptor cross-linked to a different second antigen recognition portion that binds a pathogenic antigenic molecule such that the plurality of different bispecific molecules have different second antigen recognition portions with different recognition specificities, such as with recognition specificities directed to different epitopes and/or different variants of a pathogen and/or to different pathogens. The invention also relates to methods of producing such polyclonal population of bispecific molecules. The invention further relates to methods of using such polyclonal population of bispecific molecules for the clearance of pathogens from the circulatory system of a mammal.

This application claims the benefit of U.S. Provisional Patent Application No. 60/276,200, filed on Mar. 15, 2001, which is incorporated herein by reference in its entirety.

### 1. FIELD OF THE INVENTION

The invention relates to a polyclonal population of bispecific molecules which comprises a plurality of different bispecific molecules, each of said bispecific molecules comprising a first antigen recognition portion that binds a C3b-like receptor and a different second antigen recognition portion that binds a pathogenic antigenic molecule such that the population comprises a plurality of different antigen recognition specificities. The invention also relates to methods of producing such polyclonal population of bispecific molecules. The invention further relates to methods of using such polyclonal population of bispecific molecules for the clearance of pathogens from the circulatory system of a mammal.

### 2. BACKGROUND OF THE INVENTION

Primate erythrocytes, or red blood cells (RBC's), play an essential role in the clearance of antigens from the circulatory system. The formation of an immune complex in the circulatory system activates the complement factor C3b in primates and leads to the binding of C3b to the immune complex. The C3b/immune complex then binds to the type 1 complement receptor (CR1), a C3b receptor, expressed on the surface of erythrocytes via the C3b molecule attached to the immune complex. The immune complex is then chaperoned by the erythrocyte to the reticuloendothial system (RES) in the liver and spleen for neutralization. The RES cells, most notably the fixed-tissue macrophages in the liver called Kupffer cells, recognize the C3b/immune complex and break this complex from the RBC by severing the C3b receptor-RBC junction, producing a liberated erythrocyte and a C3b/immune complex which is then engulfed by the Kupffer cells and is completely destroyed within subcellular organelles of the Kupffer cell. This pathogen clearance process, however, is complement-dependent, i.e., confined to immune complexes recognized by the C3b receptor, and is ineffective in removing immune complexes which are not recognized by the C3b receptor.

Taylor et al. have discovered a complement independent method of removing pathogens from the circulatory system. Taylor et al. have shown that chemical cross-linking of a first monoclonal antibody (mAb) specific to a primate C3b receptor to a second monoclonal antibody specific to a pathogenic antigenic molecule creates a bispecific heteropolymeric antibody which offers a mechanism for binding a pathogenic antigenic molecule to a primate's C3b receptor without complement activation. (U.S. Pat. Nos. 5,487,890; 5,470,570; and 5,879,679). It is found that the Fc portion of the anti-C3b receptor mAb plays an important role in the transfer of the erythrocyte-immune complex to an acceptor cell and the subsequent proteolysis of the erythrocyte-immune complex (Nardin et al., 1999, Molecular Immunology 36:827-835). Taylor et al. have shown that this complement-independent process can remove over 99% of pathogens from the circulation as compared to about 10-15% in naive mammals by the normal, complement-dependent, process.

However, certain pathogens are highly mutable or are heterogenous in structure or both. A bispecific molecule having a single antigen recognition specificity is normally not sufficient in the clearance of such pathogen or pathogens. For example, HIV-1 is a highly mutable virus that during the course of HIV-1 infection, the antibodies generated in an infected individual do not provide permanent protective effect due in part to the rapid emergence of neutralization escape variants (Thali et al., 1992, J. Acquired Immune Deficiency Syndromes 5:591-599). In such cases, polyclonal preparations of antibodies, such as hyperimmune anti-HIV IgG preparations obtained from the plasma of multiple infected donors, have been shown to offer certain advantages in the recognition and neutralization of a broad range of HIV isolates (Cummins et al., 1991, Blood 77:1111-1117; Lambert et al., 1997, J. Infectious Diseases 175:283-291). It is also suggested that antibodies present in such polyclonal preparation might have activities in addition to neutralization, such as binding to NK cells to effect antibody-dependent cellular cytotoxicity, which could lyse infected cells and thereby preventing cell-to-cell transmission of virus. Polyclonal hyperimmune globulin reparations for other pathogens, such as bacilli Klebsiella, have also been demonstrated (e.g., Cryz et al., 1986, J Lab Clin Med 108:182-9).

However, although providing a broad spectrum of specificities, the efficacy of polyclonal preparations can be further enhanced. More recent developments in the strategy of using polyclonal antibody populations against pathogens have also been focused on further improving the effectiveness of polyclonal antibody populations by producing populations of antibodies that are synergistic in protecting a patient against a pathogen (Laal et al., 1994, J. Virol. 68:4001-4008; Burkly et al., J. Virol. 69:4267-4273; Vijh-Warrier et al., 1996, J. Virol. 70:4466-4473; Mascola et al., 1997, J. Virol. 71:7198-7206; Schaefer et al., 2000, Infection and Immunity 68:2608-2616). For example, in the HIV-1 case, it is known that there are four major neutralization epitope clusters in the HIV-1 envelope glycoproteins, the V3 loop, the CD4 binding site, and the V2 domain in gp120 and the 2F5 mAb-defined site in gp41 (Vijh-Warrier et al., 1996, J. Virol. 70:4466-4473). Synergistic neutralization of HIV-1 virus has been demonstrated using a combination of monoclonal antibodies with specificities for two or more of these major epitope clusters, including a three mAb formulation targeting the V2 domain, the V3 loop, and the CD4 binding-site. (Laal et al., 1994, J. Virol. 68:4001-4008; Burkly et al., J. Virol. 69:4267-4273; Vijh-Warrier et al., 1996, J. Virol. 70:4466-4473; Thali et al., 1992, J. Acquired Immune Deficiency Syndromes 5:591-599) The mechanism for synergism is not yet fully understood. However, it has been found that the binding of anti-V3 loop antibodies can increase the sensitivity to neutralization by anti-CD4 antibodies, even for mutants that are resistant to neutralization by anti-CD4 antibodies (Thali et al., 1992, J. Acquired Immune Deficiency Syndromes 5:591-599), suggesting that the actions by multiple antibodies that target different epitopes may have potential benefits beyond simple additive effect. It has also been found that a combination of a polyclonal hyperimmune anti-HIV immunoglobulin preparation and two monoclonal antibodies, a monoclonal antibody inhibiting the interaction of gp120 with the b-chemokine receptor CCR5, and a monoclonal antibody affecting gp41 fusion domain shows synergistic protective effect against infection by a large panel of HIV variants (Mascola et al., 1997, J. Virol. 71:7198-7206). The synergism in this case is suggested to be a consequence of the complementary activity of the two mAbs and the functionally diverse spectrum of the hyperimmune anti-HIV immunoglobulin preparation. This approach also has the additional advantage of being more adaptable in that the composition of the polyclonal population can be adjusted to maximize protection under specific conditions, for example, tailored according to specific needs of particular patients or population of patients.

Therefore, to fully exploit the potential of using bispecific molecules in removing pathogens, there is a need to produce populations of bispecific molecules that comprises a plurality of specificities directed to, e.g., multiple epitopes on a targeted pathogen and/or multiple variants of a targeted pathogen. By targeting multiple epitopes and/or multiple variants of a pathogen, the polyclonal population of bispecific molecules is also advantageous in the clearance of pathogens that have a higher mutation rate because simultaneous mutations at more than one epitopes tend to be much less frequent. Such populations of bispecific molecules can also be used for targeting and removing heterogenous mixture of pathogens.

Discussion or citation of a reference herein shall not be construed as an admission that such reference is prior art to the present invention.

### 3. SUMMARY OF THE INVENTION

The present invention relates to a polyclonal population of bispecific molecules which comprises a plurality of different bispecific molecules, each of said bispecific molecules comprising a first antigen recognition portion that binds a C3b-like receptor cross-linked to a different second antigen recognition portion that binds a pathogenic antigenic molecule. The population thus comprises a plurality of different antigen recognition specificities, e.g., directed to different epitopes and/or different variants of a pathogen or pathogens and/or pathogenic antigenic molecule or pathogenic antigenic molecules. The polyclonal population of bispecific molecules are produced by cross-linking a first antigen recognition portion that binds a C3b-like receptor to each member of a polyclonal collection of second antigen recognition portions that comprises a plurality of different specificities.

The first antigen recognition portion in a bispecific molecule in the plurality of bispecific molecules in the polyclonal population of the present invention can be any molecule or fragment thereof comprising a C3b-like receptor binding domain and preferably an effector domain. In a preferred embodiment, the first antigen recognition portion comprises an anti-CR1 monoclonal antibody. The first antigen recognition portion can also be a single chain Fv fragment fused to an Fc domain or a chimeric antibody comprising a C3b-like receptor binding domain and an effector domain.

The second antigen recognition portion of a bispecific molecule in the plurality of bispecific molecules in the polyclonal population of the present invention can be any molecular moiety that recognizes and binds a pathogenic antigenic molecule, including but is not limited to any antibody or antigen binding fragment thereof. The second antigen recognition portion can also be any molecular moiety that is recognized and bound by a pathogenic antigenic molecule to be cleared. The polyclonal population of the invention comprises a plurality of different bispecific molecules having different second antigen recognition portions that have specificities directed to, e.g., a plurality of recognition sites on a pathogen and/or pathogens. As a non-limiting example, the population of bispecific molecules can have a plurality of different second antigen recognition portions that recognize and bind different epitopes on a pathogen. The population of bispecific molecules can also have a plurality of different second antigen recognition portions that recognize and bind the same epitope on a pathogen.

In preferred embodiments of the invention, each bispecific molecule in the plurality of bispecific molecules in the polyclonal population of the present invention comprises an anti-CR1 mAb cross-linked to a different second antigen recognition portion that recognizes and binds an antigenic pathogenic molecule of interest. In more preferred embodiments of the invention, each bispecific molecule in the plurality of bispecific molecules in the polyclonal population of the present invention comprises an anti-CR1 mAb cross-linked to a different mAb that recognizes and binds an antigenic pathogenic molecule of interest. In still more preferred embodiments, each bispecific molecule in the plurality of bispecific molecules in the polyclonal population of the present invention comprises an anti-CR1 mAb cross-linked to a different immunoglobulin molecule that recognizes and binds an antigenic pathogenic molecule of interest

The characteristic and function of each member bispecific molecule in the plurality of bispecific molecules in the polyclonal population can be known or unknown. The exact proportion of each member bispecific molecule in the plurality of bispecific molecules in the polyclonal population can also be known or unknown. Preferably, the characteristics and the proportions of at least some member bispecific molecules in the plurality of bispecific molecules in the polyclonal population are known so that if desired, the exact proportions of such members can be adjusted for optimal therapeutic and/or prophylactic efficacy. The polyclonal population of bispecific molecules can comprise bispecific molecules that do not bind the target pathogenic antigenic molecule or pathogenic antigenic molecules. For example, the population of bispecific molecules can be prepared from a hyperimmune serum that contains antibodies that bind antigenic molecules other than those on the target pathogens. Preferably, the plurality of bispecific molecules in the polyclonal population constitutes at least 10%, 20%, 50% or 80% of the population. More preferably, the plurality of bispecific molecules in the polyclonal population constitutes at least 90% of the population. The plurality of ispecific molecules in the polyclonal population of ispecific molecules preferably does not comprise any single ispecific molecule which has a proportion exceeding 90%, 80%, or 50% of the plurality. More preferably, the plurality of bispecific molecules in the polyclonal population of bispecific molecules does not comprise any single bispecific molecule which has a proportion exceeding 20% of the plurality. The plurality of bispecific molecules in the polyclonal population comprises at least 2 different bispecific molecules with different antigen recognition specificities. Preferably, the plurality of bispecific molecules in the polyclonal population comprises at least 10 different bispecific molecules with different antigen recognition specificities. More preferably, the plurality of bispecific molecules in the polyclonal population comprises at least 100 different bispecific molecules with different antigen recognition specificities. The polyclonal population can be generated from a suitable polyclonal population of antigen recognition portions, such as but not limited to a polyclonal immunoglobulin preparation. Preferably, each bispecific molecule in the polyclonal population does not inhibit or impair other bispecific molecule's activity. More preferably, one or more bispecific molecules in the polyclonal population are synergistic with the plurality bispecific molecules in the polyclonal population in pathogen neutralization. Most preferably, one or more bispecific molecules in the polyclonal population enhance the effectiveness of one or more other bispecific molecule(s).

In another embodiment, the invention provides a population of modified hematopoietic cells that consists essentially of a population of hematopoietic cells each bound to one or more bispecific molecules, wherein each of said bispecific molecules comprises a first antigen recognition portion that binds a C3b-like receptor cross-linked to a different second antigen recognition portion that binds a pathogenic antigenic molecule, wherein said bispecific molecules bound to said population of modified hematopoietic cells forms a population of bispecific molecules comprising different second antigen recognition portions.

The polyclonal population of bispecific molecules of the invention is prepared by a method comprising cross-linking a population of anti-CR1 portions and a polyclonal population of antigen recognition portions by a chemical cross-linking agent. Any standard chemical cross-linking methods can be used in the present invention. For example, cross-linking agents, including but are not limited to, protein A, glutaraldehyde, carbodiimide, N-succinimidyl-S-acetyl-thioacetate (SATA), N-succinimidyl-3-(2-pyridyldithio)propionate (SPDP), and sulfosuccinimidyl 4-(N-maleimidomethyl) cyclohexane-1-carboxylate (sulfo-SMCC) can be used. In a preferred embodiment, cross-linking agents N-succinimidyl S-acetylthioacetate (SATA) and sulfosuccinimidy 4-(N-maleimidomethyl)cyclohexane-1-carboxylate (sulfo-SMCC) are used to cross-link a population of anti-CR1 portions and a polyclonal population of antigen recognition portions. In another preferred embodiment, N-succinimidyl 3-(2-pyridyldithio)propionate (SPDP) is used to cross-link a population of anti-CR1 portions and a polyclonal population of antigen recognition portions.

The invention provides methods for treating a mammal having a disease or disorder or undesirable condition associated with the presence of one or more pathogens. The methods of the invention comprise administering to the mammal a therapeutically effective dose of a polyclonal population of bispecific molecules comprising a plurality of different bispecific molecules, each bispecific molecule in the plurality comprising a first antigen recognition portion that binds a C3b-like receptor cross-linked to a different second antigen recognition portion that binds the pathogenic antigenic molecule or pathogenic antigenic molecules such that the polyclonal population comprises a plurality of different bispecific molecules having a plurality of antigen recognition specificities directed to, e.g., a plurality of recognition sites on the pathogen or pathogenic antigenic molecule or to a plurality of variants of the pathogens or pathogenic antigenic molecules. The invention also provides methods for preventing a disease or disorder or undesirable condition associated with the presence of one or more pathogens in a mammal. The methods comprise administering, prior to the onset of said disease or disorder or undesirable condition, to the mammal a prophylactically effective dose of a polyclonal population of bispecific molecules comprising a plurality of different bispecific molecules, each bispecific molecule in the plurality comprising a first antigen recognition portion that binds a C3b-like receptor cross-linked to a different second antigen recognition portion that binds the pathogenic antigenic molecule or pathogenic antigenic molecules such that the polyclonal population comprises a plurality of different bispecific molecules having a plurality of antigen recognition specificities directed to, e.g., a plurality of recognition sites on the pathogen or pathogenic antigenic molecule or to a plurality of variants of the pathogens or pathogenic antigenic molecules.

### 4. BRIEF DESCRIPTION OF FIGURES

FIG. 1A is a schematic illustration of a bispecific molecule in the polyclonal population; FIG. 1B is a schematic illustration of a polyclonal population of bispecific molecules; and FIG. 1C is a schematic illustration of activities of the polyclonal population of bispecific molecules as compared to a monoclonal bispecific molecule.

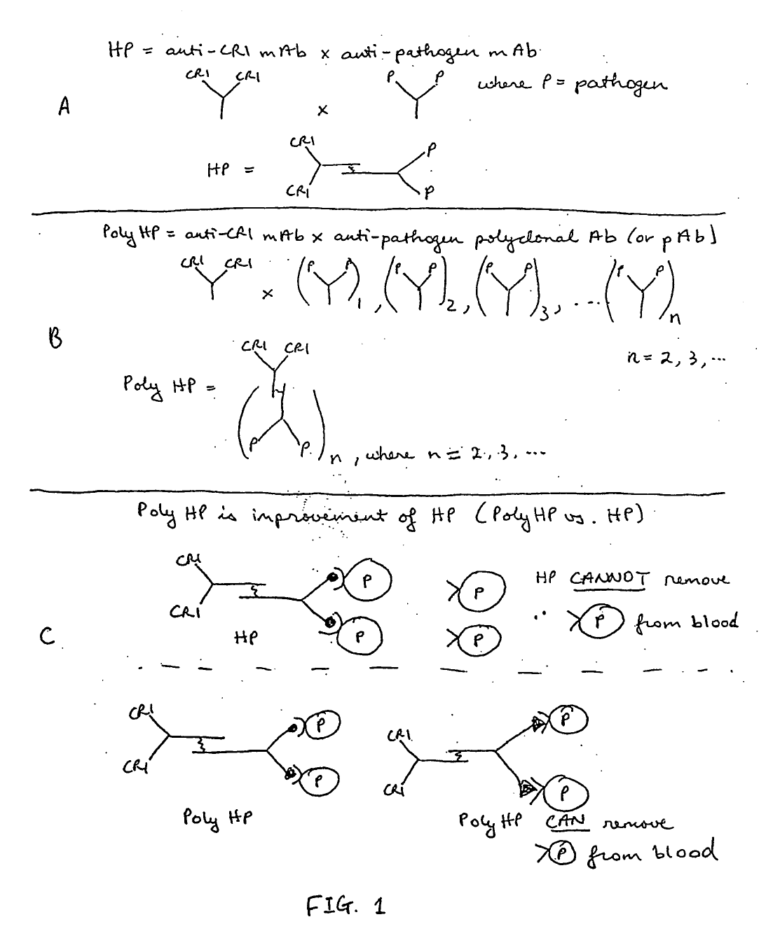

### 5. DETAILED DESCRIPTION OF THE INVENTION

The present invention relates to a polyclonal population of bispecific molecules which comprises a plurality of bispecific molecules, each of said bispecific molecules comprising a first antigen recognition portion that binds a C3b-like receptor cross-linked to a different second antigen recognition portion that binds a pathogenic antigenic molecule. The population thus comprises a plurality of different bispecific molecules having a plurality of antigen recognition specificities directed to different pathogenic antigenic molecules, e.g., different epitopes and/or different variants of a pathogen or pathogens. The polyclonal population of bispecific molecules is produced by cross-linking a first antigen recognition portion that binds a C3b-like receptor to each member of a polyclonal population of different second antigen recognition portions having a plurality of different specificities. The invention also relates to methods for the production of the polyclonal population of bispecific molecules by chemical cross-linking methods. The invention further relates to methods of using the polyclonal population of bispecific molecules of the present invention for the clearance of pathogen or pathogens and/or pathogenic antigenic molecule or pathogenic antigenic molecules, such as pathogens that have multiple epitopes and/or multiple variants as well as heterogenous mixture of pathogens or pathogenic antigenic molecules.

A bispecific molecule generally refers to a molecule having two or more different antigen recognition specificities. The bispecific molecule of the present invention refers to a molecule having a first antigen recognition portion that binds a C3b-like receptor, such as the type 1 complement receptor in primates, and a second antigen recognition portion that binds a pathogenic antigenic molecule, such as but is not limited to an epitope of a pathogen, to be cleared from the circulation. In the present invention, the first and second antigen recognition portions are linked by chemical cross-linker(s).

As used herein, the term “antigen recognition portion” refers to a molecular moiety that recognizes and binds a pathogenic antigenic molecule or, alternatively, is recognized and bound by a pathogenic antigenic molecule. The molecular moiety can contain antigen binding domain(s) and effector domain(s). An effector domain refers to a portion of the molecular moiety that facilitates the transfer of the blood cell-immune complex, such as the erythrocyte-immune complex in primate, to an acceptor cell for proteolysis. For example, an effector domain can comprise an Fc domain of an mAb, i.e., a hinge region, a CH2 domain and a CH3 domain of a heavy chain. Alternatively, an effector domain can comprise an Fc domain with the CH2 domain and the CH3 domain in reverse order, i.e., the CH3 domain appears at the amino terminal side of the CH2 domain.

As used herein, the term “molecular moiety” encompasses any molecule or fragment thereof, including but are not limited to peptides and polypeptides, nucleic acids, oligosaccharide, organic small molecules, and any combination thereof.

As used herein, the term “C3b-like receptor” refers to any mammalian circulatory molecule expressed on the surface of a mammalian blood cell, which has an analogous function to a primate C3b receptor, the CR1, in that it binds to a molecule associated with an immune complex, which is then chaperoned by the blood cell to, e.g., a phagocytic cell for clearance. A mammalian blood cell can be, but is not limited to, a primate red-blood cell or erythrocyte.

As used herein, a polyclonal population of bispecific molecules of the present invention refers broadly to any population comprising a plurality of different bispecific molecules, each of which comprising a first antigen recognition portion that binds a C3b-like receptor cross-linked to a different second antigen recognition portion that binds a pathogenic antigenic molecule. The population thus comprises a plurality of different bispecific molecules having a plurality of different antigen recognition specificities. The plurality of different second antigen recognition portions can recognize and bind the same epitope on a pathogen. The plurality of different antigen recognition specificities can also be directed to a plurality of different epitopes on a pathogen. When the plurality of different antigen recognition specificities is directed to a plurality of different epitopes on a pathogen, it is preferred that at least some member bispecific molecules in the polyclonal population act synergistically. The plurality of different antigen recognition specificities can also be directed to a plurality of variants of a pathogen. The plurality of different antigen recognition specificities can further be directed to a plurality of different pathogens. The plurality of different antigen recognition of specificities can further be directed to a plurality of different epitopes on a plurality of different pathogens. The characteristic and function of each member bispecific molecule in the plurality of bispecific molecules in the polyclonal population can be known or unknown. The exact proportion of each member bispecific molecule in the plurality of bispecific molecules in the polyclonal population can also be known or unknown. Preferably, the characteristics and the proportions of at least some member bispecific molecules in the plurality of bispecific molecules in the polyclonal population are known so that if desired, the exact proportions of such members can be adjusted for optimal therapeutic and/or prophylactic efficacy. The polyclonal population of bispecific molecules can comprise bispecific molecules that do not bind the target pathogenic antigenic molecule or pathogenic antigenic molecules. For example, the population of bispecific molecules can be prepared from a hyperimmune serum that contains antibodies that bind antigenic molecules other than those that are on the target pathogens. Preferably, the plurality of bispecific molecules in the polyclonal population constitutes at least 1%, 5%, 10%, 20%, 50% or 80% of the population. More preferably, the plurality of bispecific molecules in the polyclonal population constitutes at least 90% of the population. The plurality of bispecific molecules in the polyclonal population of bispecific molecules preferably does not comprise any single bispecific molecule which has a proportion exceeding 95%, 80%, or 60% of the plurality. More preferably, the plurality of bispecific molecules in the polyclonal population of bispecific molecules does not comprise any single bispecific molecule which has a proportion exceeding 50% of the plurality. The plurality of bispecific molecules in the polyclonal population comprises at least 2 different bispecific molecules with different antigen recognition specificities. Preferably, the plurality of bispecific molecules in the polyclonal population comprises at least 10 different bispecific molecules with different antigen recognition specificities. More preferably, the plurality of bispecific molecules in the polyclonal population comprises at least 100 different bispecific molecules with different antigen recognition specificities. The polyclonal population can be a polyclonal population generated from a suitable polyclonal population of antigen recognition portions, such as but is not limited to a polyclonal immunoglobulin preparation.

As used herein, “epitope” refers to an antigenic determinant, i.e., a region of a molecule that provokes an immunological response in a host or is bound by an antibody. This region can but need not comprise consecutive amino acids. The term epitope is also known in the art as “antigenic determinant.” An epitope may comprise as few as three amino acids in a spatial conformation which is unique to the immune system of the host. Generally, an epitope consists of at least five such-amino acids, and more usually consists of at least 8-10 such amino acids. Methods for determining the spatial conformation of such amino acids are known in the art.

As used herein, “synergistic therapeutic or prophylactic effect” means that the inclusion of one or more bispecific molecules in the polyclonal population of bispecific molecules leads to synergistic effects in neutralization of a pathogen or a pathogenic antigenic molecule, i.e., the combination has neutralizing capacity beyond simple additive effects. Preferably, each member in the polyclonal population does not compete with other members for binding sites and/or inhibit or impair other member's activity in neutralization of a pathogen or pathogenic antigenic molecule. More preferably, the action of one member of the polyclonal population enhances the effectiveness of other member(s).

### 5.1. Bispecific Molecules

In the present invention, the polyclonal population of bispecific molecules comprises a plurality of bispecific molecules each comprising a first antigen recognition portion that binds a C3b-like receptor cross-linked to a second antigen recognition portion that binds a pathogenic antigenic molecule.

The first antigen recognition portion of a bispecific molecule in the plurality of bispecific molecules in the polyclonal population of the present invention can be any polypeptide that contains a C3b-like receptor binding domain and preferably an effector domain. In a preferred embodiment, the first antigen recognition portion is an anti-CR1 mAb. In another embodiment, the first antigen recognition portion is an anti-CR1 polypeptide antibody, including but is not limited to, a single-chain variable region fragment (scFv) with specificity for a C3b-like receptor fused to the N-terminus of an immunoglobulin Fc domain. The first antigen recognition portion can also be a chimeric antibody, such as but is not limited to a humanized monoclonal antibody wherein the complementarity determining regions are from a non-human species, e.g., mouse, and the framework regions are human thereby decreasing the likelihood of an immune response in human patients treated with the antibody (U.S. Pat. Nos. 4,816,567, 4,816,397, 5,693,762; 5,585,089; 5,565,332 and 5,821,337 which are incorporated herein by reference in their entirety). The chimeric antibody can also have non-human constant domain replaced with human constant domain. Preferably, the Fc domain of the chimeric antibody can be recognized by the Fc receptors on phagocytic cells of the intended patients, thereby facilitating the transfer and subsequent proteolysis of the RBC-immune complex. Although, for simplicity, this disclosure often makes references to an anti-CR1 antigen recognition portion or an anti-CR1 antibody, it is understood that such antigen recognition portion or antibody refers to an antigen recognition portion or antibody that binds a C3b-like receptor.

The second antigen recognition portion of the bispecific molecule in the plurality of bispecific molecules in the polyclonal population of the present invention can be any molecule or fragment thereof that recognizes and binds a pathogenic antigenic molecule, e.g., a naturally occurring antigen, and/or any derivative or fragment thereof. The pathogenic antigenic molecule can be any substance that is present in the circulation that is potentially injurious to or undesirable in the subject to be treated, including but is not limited to proteins or drugs or toxins, autoantibodies or autoantigens, or a molecule of any infectious agent or its products. A pathogenic antigenic molecule is any molecule containing an antigenic determinant (or otherwise capable of being bound by a binding domain) that is or is part of a substance (e.g., a pathogen) that is the cause of a disease or disorder or any other undesirable condition. The second antigen recognition portion of the invention can be any type of molecule, including but is not limited to peptide and polypeptide, nucleic acid, oligosaccharide, and organic small molecule. The polyclonal population of the invention comprises a plurality of different second antigen recognition portions that have specificities directed to a plurality of different recognition sites of a pathogen and/or a plurality of different pathogens. As a non-limiting example, the population of bispecific molecules can have a plurality of different second antigen recognition portions that recognize and bind different epitopes on a pathogen. However, when desired, the population of bispecific molecules can also have a plurality of different second antigen recognition portions that recognize and bind the same epitope on a pathogen or pathogenic antigenic molecule.

In preferred embodiments of the invention, each bispecific molecule in the plurality of bispecific molecules in the polyclonal population of the present invention comprises an anti-CR1 mAb cross-linkded to a different second antigen recognition portion. The second antigen recognition portion can be, but are not limited to, an antigen binding domain, an epitope, a nucleic acid, or an organic small molecule. In more preferred embodiments, each bispecific molecule in the plurality of bispecific molecules in the polyclonal population of the invention comprises an anti-CR1 mAb cross-linked to an mAb that recognizes and binds an antigenic molecule, such as but is not limited to an epitope of a pathogen.

In another embodiment of the invention, each bispecific molecule in the plurality of bispecific molecules in the polyclonal population of the present invention comprises an anti-CR1 polypeptide antibody, including but is not limited to, a scFv with specificity for a C3b-like receptor fused to the N-terminus of an immunoglobulin Fc domain, cross-linked to an antigen recognition portion. The antigen recognition portion can be, but is not limited to, an antigen binding domain (e.g., an antigen binding mAb), an epitope, a nucleic acid, or an organic small molecule.

### 5.2. Polyclonal Populations of Bispecific Molecules

In the present invention, a polyclonal population of bispecific molecules comprises a plurality of bispecific molecules having a plurality of different antigen recognition specificities as described in Section 5.1. The plurality of different antigen recognition specificities can be directed to a plurality of different epitopes of the same pathogen. When the plurality of different antigen recognition specificities is directed to a plurality of different epitopes on the same pathogen or pathogenic antigenic molecule, it is preferred that some bispecific molecules in the polyclonal population act synergistically in neutralization of the pathogen or the pathogenic antigenic molecule with bispecific molecules in the plurality of bispecific molecules in the polyclonal population. The plurality of different antigen recognition specificities can also be directed to a plurality of variants of a pathogen and/or a plurality of different pathogens. The plurality of different antigen recognition specificities can further be directed to a plurality of different epitopes on a plurality of different variants of a pathogen and/or a plurality of different pathogens.

The characteristic and function of each bispecific molecule in the plurality of bispecific molecules in the polyclonal population can be known or unknown. Preferably the characteristics and functions of at least some bispecific molecules in the plurality are known so that if desired, the proportions of such bispecific molecules in the plurality can be adjusted for optimal therapeutic and/or prophylactic efficacy. More preferably, the characteristic and function of each bispecific molecule in the plurality of bispecific molecules are known. Preferably, each bispecific molecule in the plurality does not inhibit or impair the function of other bispecific molecules. As a non-limiting example, the binding of a bispecific molecule in the population to its binding eptiope does not cause conformation changes in other, different epitopes that may reduce the binding affinity of other, different bispecific molecule or molecules in the population. Most preferably, the activity of one or more bispecific molecules in the population is synergistic in neutralization of the pathogen or pathogenic antigenic molecule of interest in the plurality of bispecific molecules is synergistic with that of one or more bispecific molecules in the plurality of bispecific molecules in the population.

The exact proportion of bispecific molecules in the plurality of bispecific molecules in the polyclonal population can also be known or unknown. Preferably the exact proportion of bispecific molecules in the plurality of bispecific molecules in the polyclonal population are known so that if desired, the population can be adjusted for optimal therapeutic and/or prophylactic efficacy. The polyclonal population of bispecific molecules can comprise bispecific molecules that do not bind the target pathogenic antigenic molecule or pathogenic antigenic molecules. For example, the population of bispecific molecules can be prepared from a hyperimmune serum that contains antibodies that bind antigenic molecules other than those on the target pathogens. Preferably, the plurality of bispecific molecules in the polyclonal population constitutes at least 1%, 5%, 10%, 20%, 50% or 80% of the population. More preferably, the plurality of bispecific molecules in the plurality of bispecific molecules in the polyclonal population constitutes at least 90% of the population. The plurality of bispecific molecules in the polyclonal population of bispecific molecules preferably does not comprise any single bispecific molecule which has a proportion exceeding 95%, 80%, or 60% of the plurality of bispecific molecules in the population. More preferably, the plurality of bispecific molecules in the polyclonal population of bispecific molecules does not comprise any single bispecific molecule which has a proportion exceeding 50% of the plurality of bispecific molecules in the population. The plurality of bispecific molecules in the polyclonal population comprises at least 2 different bispecific molecules with different antigen recognition specificities. Preferably, the plurality of bispecific molecules in the polyclonal population comprises at least 10 different bispecific molecules with different antigen recognition specificities. More preferably, the plurality of bispecific molecules in the polyclonal population comprises at least 100 different bispecific molecules with different antigen recognition specificities.

The polyclonal population of bispecific molecules can be produced from a polyclonal immunoglobulin preparation. The polyclonal population of bispecific molecules can also be a polyclonal library of bispecific molecules produced from a suitable polyclonal library of antigen recognition portions. The polyclonal population of bispecific molecules can further be produced from a cocktail of different monoclonal antigen recognition portions. The bispecific molecules in a population of bispecific molecules can be produced using any of the chemical cross-linking methods know in the art.

### 5.3. Methods of Producing Polyclonal Populations of Bispecific Molecules

The polyclonal population of bispecific molecules of the present invention is produced by cross-linking a polyclonal population of antigen recognition portions that bind a pathogenic antigenic molecule or pathogenic antigenic molecules to a population of antigen recognition portions that bind a C3b-like receptor. In preferred embodiments, the entire polyclonal population of bispecific molecules can be produced in one reaction. Such can normally be done by first producing a polyclonal population of antigen recognition portions and cross-linking the entire population of such antigen recognition portions to a population of C3b-like receptor binding portions without isolation of individual members. In other preferred embodiments, members and/or fractions of the polyclonal population can be produced separately and then combined to form the polyclonal population. Such embodiments are useful when polyclonal populations with specific compositions are to be produced.

### 5.3.1. Production of Anti-CR1 Portion

In preferred embodiments, the anti-CR1 portion of the bispecific molecule comprises an anti-CR1 mAb. An anti-CR1 mAb that binds a human C3b receptor can be produced by known methods. In one embodiment, anti-CR1 mAb, preferably an anti-CR1 IgG, can be prepared using standard hybridoma precedure known in the art (see, for example, Kohler and Milstein, 1975, Nature 256:495-497; Hogg et al., 1984, Eur. J. Immunol. 14:236-243; O'Shea et al., 1985, J. Immunol. 134:2580-2587; Schreiber, U.S. Pat. No. 4,672,044). A suitable mouse is immunized with human CR1 which can be purified from human erythrocytes. The spleen cells obtained from the immunized mouse are fused with an immortal mouse myeloma cell line which results in a population of hybridoma cells, including a hybridoma that produces an anti-CR1 antibody. The hybridoma which produces the anti-CR1 antibody is then selected, or ‘cloned’, from the population of hybridomas using conventional techniques such as enzyme linked immunosorbent assays (ELISA). Hybridoma cell lines expressing anti-CR1 mAb can also be obtained from various sources, for example, the murine anti-CR1 mAb that binds human CR1 described in U.S. Pat. No. 4,672,044 is available as hybridoma cell line ATCC HB 8592 from the American Type Culture Collection (ATCC). The obtained hybridoma cells are grown and washed using standard methods known in the art. Anti-CR1 antibodies are then recovered from supernatants.

In other embodiments, nucleic acids encoding the heavy and light chains of an anti-CR1 mAb, preferably an anti-CR1 IgG, are prepared from the hybridoma cell line by standard methods known in the art. As a non-limiting example, cDNAs encoding the heavy and light chains of the anti-CR1 IgG are prepared by priming mRNA using appropriate primers, followed by PCR amplification using appropriate forward and reverse primers. Any commercially available kits for cDNA synthesis can be used. The nucleic acids are used in the construction of expression vector(s). The expression vector(s) are transfected into a suitable host. Non-limiting examples include E. coli, yeast, insect cell, and mammalian systems, such as a Chinese hamster ovary cell line. Antibody production can be induced by standard method known in the art.

In another embodiments, anti-CR1 scFv's are prepared according to standard methods known in the art. In one embodiment, anti-CR1 chimeric antibodies and nucleic acids encoding such anti-CR1 chimeric antibodies are prepared according to standard methods known in the art (U.S. Pat. Nos. 4,816,567, 4,816,397, 5,693,762; 5,585,089; 5,565,332 and 5,821,337 which are incorporated herein by reference in their entirety).

Anti-CR1 antigen recognition portions can also be produced by standard phage display technologies. Kits for generating and screening phage display libraries are commercially available (e.g., Pharmacia Recombinant Phage Antibody System, Catalog No. 27-9400-01; and the Stratagene antigen SurfZAPT™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

The anti-CR1 portion can then be used in the production of the polyclonal population of bispecific molecules.

### 5.3.2. Production and Purification of Polyclonal Populations of Antigen Recognition Portions

Polyclonal populations of antigen recognition portions having specificities directed to a plurality of recognition sites on a pathogen and/or pathogens can be produced by any methods known in the art. For example, methods for preparing a hyperimmune serum preparation against a pathogen can be used in the present invention.

### 5.3.2.1. Production by Immunization of Animals

The polyclonal population of antigen recognition portions can be produced by immunization of a suitable animal, such as but are not limited to mouse, rabbit, and horse.

An immunogenic preparation, typically comprising the antigenic molecules, e.g., associated with the pathogen or pathogens to be cleared from a subject, are used to prepare antibodies by immunizing a suitable subject (e.g., rabbit, goat, mouse or other mammal). An appropriate immunogenic preparation can contain, for example, recombinantly expressed or chemically synthesized antigen. The preparation can further include an adjuvant, such as Freund's complete or incomplete adjuvant, or similar immunostimulatory agent.

Isolated antigens to be used as immunogens, as well as isolated antigenic fragments, are suitable for use as immunogens to raise antibodies directed against an antigen. An isolated antigenic fragment suitable for use as an immunogen comprises at least a portion of the antigen that is 8 amino acids, more preferably 10 amino acids and more preferably still, 15 amino acids long.

In another embodiment, the antigen for use as an immunogen can be isolated from cells or tissue sources by an appropriate purification scheme using standard purification techniques. In another embodiment, immunogenic antigens are produced by recombinant DNA techniques. Alternative to recombinant expression, an antigen can be synthesized chemically using standard peptide synthesis techniques.

An “isolated” antigen is substantially free of cellular material or other contaminating material from the cell or tissue source from which the protein is derived, or substantially free of chemical precursors or other chemicals when chemically synthesized. The language “substantially free of cellular material” includes preparations of antigen in which the antigen is separated from cellular components of the cells from which it is isolated or recombinantly produced. Thus, an antigen that is substantially free of cellular material includes preparations of antigen having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the antigen. Accordingly such preparations of the antigen have less than about 30%, 20%, 10%, 5% (by dry weight) of chemical precursors or compounds other than the polypeptide of interest.

The invention also provides chimeric or fusion antigens for use as immunogens. As used herein, a “chimeric antigen” or “fusion antigen” comprises all or part of an antigen for use in the invention, operably linked to a heterologous polypeptide. Within the fusion antigen, the term “operably linked” is intended to indicate that the antigen and the heterologous polypeptide are fused in-frame to each other. The heterologous polypeptide can be fused to the N-terminus or C-terminus of the antigen.

One useful fusion antigen is a GST fusion antigen in which the antigen is fused to the C-terminus of GST sequences. Such fusion antigens can facilitate the purification of a recombinant antigens.

In another embodiment, the fusion antigen contains a heterologous signal sequence at its N-terminus so that the antigen can be secreted and purified to high homogeneity in order to produce high affinity antibodies. For example, the native signal sequence of an immunogen can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla; Calif.). In yet another example, useful prokaryotic heterologous signal sequences include the phoA secretory signal and the protein A secretory signal (Pharmacia Biotech; Piscataway, N.J.).

In yet another embodiment, the fusion antigen is an immunoglobulin fusion protein in which all or part of an antigen is fused to sequences derived from a member of the immunoglobulin protein family. The immunoglobulin fusion proteins can be used as immunogens to produce antibodies directed against an antigen in a subject and to potentially purify additional antigens.

Chimeric and fusion proteins can be produced by standard recombinant DNA techniques. In one embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (e.g., Ausubel et al., supra). Moreover, many expression vectors are commercially available that already encode a fusion domain (e.g., a GST polypeptide). A nucleic acid encoding an immunogen can be cloned into such an expression vector such that the fusion domain is linked in-frame to the polypeptide.

In still other embodiments of the invention, a mixture of toxic substances, such as those contained in a reptile or snake bite, is obtained.

The immunogen is then used to immunize a suitable animal. Preferably, the animal is a specialized transgenic animal that can secret human antibody. Non-limiting examples include transgenic mouse strains which can be used to produce a polyclonal population of antibodies directed to a specific pathogen (Fishwild et al., 1996, Nature Biotechnology 14:845-851; Mendez et al., 1997, Nature Genetics 15:146-156). In one embodiment of the invention, transgenic mice that harbor the unrearranged human immunoglobulin genes are immunized with the target immunogens. Afer a vigorous immune response against the immunogen has been elicited in the mice, the blood of the mice are collected and a purified preparation of human IgG molecules can be produced from the plasma or serum. Any methods known in the art can be used to obtain the purified preparation of human IgG molecules, including but is not limited to affinity column chromatography using anti-human IgG antibodies bound to a suitable column matrix. Anti-human IgG antibodies can be obtained from any sources known in the art, e.g., from commercial sources such as Dako Corporation and ICN. The preparation of IgG molecules produced comprises a polyclonal population of IgG molecules that bind to the immunogen or immunogens at different degree of affinity. Preferably, a substantial fraction of the preparation are IgG molecules specific to the immunogen or immunogens. Although polyclonal preparations of IgG molecules are described, it is understood that polyclonal preparations comprising any one type or any combination of different types of immunoglobulin molecules are also envisioned and are intended to be within the scope of the present invention.

The purified polyclonal preparation can then be used in the production of the polyconal population of bispecific molecules.

### 5.3.2.2. Production from Human Donors

A polyclonal preparation of antibodies or hyperimmune serum directed to a specific pathogen or pathogens and/or pathogenic antigenic molecule or pathogenic antigenic molecules can be produced from human patients who have been infected by the pathogen or pathogens and/or the pathogenic antigenic molecule or pathogenic antigenic molecules using any methods known in the art (see, e.g., Harlow et al., Using Antibodies A Laboratory Manual). As non-limiting examples, hyperimmune serum against parasites, bacteria, and viruses can be prepared according to methods described in, e.g., Shi et al., 1999, American J Tropical Med. Hyg. 60:135-141, Cryz et al., 1986, J. Lab. Clin. Med. 108:182-189, and Cummins et al., 1991, Blood 77:1111-1117.

In a preferred embodiment, a polyclonal human IgG preparation is produced using a chromatographic method as described in Tanaka et al., 1998, Brazilian Journal of Medical and Biological Research 31:1375-81, which is incorporated herein by reference in its entirety. Specifically, a combination of ion-exchange, DEAE-Sepharose FF and arginine Sepharose 4B affinity chromatography, and Sephacryl S-300 HR gel filtration is used to produce purified IgG molecules from the gamma-globulin fraction of the human plasma.

However, the present invention is not limited to polyclonal preparations of IgG molecules. It is understood that polyclonal preparations comprising any one type or any combination of different types of immunoglobulin molecules, including but are not limited to IgG, IgE, IgA, etc., are also envisioned and are intended to be within the scope of the present invention. Such polyclonal preparations can be produced using any standard method known in the art.

### 5.3.2.3. Production from Phage Display Libraries

A population of antibodies directed to a specific pathogenic antigenic molecule or pathogenic antigenic molecules can be produced from a phage display library. Polyclonal antibodies can be obtained by affinity screening of a phage display library having a sufficiently large and diverse population of specificities with an antigen or antigens of interest. Examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734. A phage display library permits selection of desired antibody or antibodies from a very large population of specificities. An additional advantage of a phage display library is that the nucleic acids encoding the selected antibodies can be obtained conveniently, thereby facilitating subsequent construction of expression vectors.

In a preferred embodiment, the polyclonal population of antibodies directed to a pathogenic antigenic molecule or pathogenic antigenic molecules is produced from a phage display library according to Den et al., 1999, J. Immunol. Meth. 222:45-57; Sharon et al. Comb. Chem. High Throughput Screen. 2000 3:185-96; and Baecher-Allan et al., Comb. Chem. High Throughput Screen. 2000 2:319-325. The phage display library is screened to select a polyclonal sublibrary having binding specificities directed to the antigenic molecule or antigenic molecules of interests by affinity chromatography (McCafferty et al., 1990, Nature 248:552; Breitling et al., 1991, Gene 104:147; and Hawkins et al., 1992, J. Mol. Biol. 226:889). The nucleic acids encoding the heavy and light chain variable regions are then linked head to head to generate a library of bidirectional phage display vectors. The bidirectional phage display vectors are then transferred in mass to bidirectional mammalian expression vectors (Sarantopoulos et al., 1994,J. Immunol. 152:5344) which are used to transfect a suitable hybridoma cell line. The transfected hybridoma cells are induced to produce the antibodies using any method known in the art.

In other preferred embodiments, the population of antibodies directed to a pathogenic antigenic molecule or pathogenic antigenic molecules is produced by a method using the whole collection of selected displayed antibodies without clonal isolation of individual members as described in U.S. Pat. No. 6,057,098, which is incorporated by reference herein in its entirety. Polyclonal antibodies are obtained by affinity screening of a phage display library having a sufficiently large repertoire of specificities with, e.g., an antigenic molecule having multiple epitopes, preferably after enrichment of displayed library members that display multiple antibodies. The nucleic acids encoding the selected display antibodies are excised and amplified using suitable PCR primers. The nucleic acids can be purified by gel electrophoresis such that the full length nucleic acids are isolated. Each of the nucleic acids is then inserted into a suitable expression vector such that a population of expression vectors having different inserts is obtained. The population of expression vectors is then expressed in a suitable host.

### 5.3.2.4. Production by Combining Monoclonal Antibodies

A population of antibodies directed to a specific pathogen or pathogens and/or pathogenic antigenic molecule or pathogenic antigenic molecules can be produced from combining different monoclonal antibodies with specificities directed to the pathogen or pathogens and/or pathogenic antigenic molecule or pathogenic antigenic molecules.

Monoclonal antibodies can be prepared by immunizing a suitable subject with an antigen as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique by Kozbor et al. (1983, Immunol. Today 4:72), the EBV-hybridoma technique by Cole et al. (1985, Monoclonal Antibodies and Cancer Therapy, Alan R. Liss, Inc., pp. 77-96) or trioma techniques. The technology for producing hybridomas is well known (see generally Current Protocols in Immunology, 1994, John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., 1975, Nature, 256:495, or may be made by recombinant DNA methods (U.S. Pat. No. 4,816,567). The term “monoclonal antibody” as used herein also indicates that the antibody is an immunoglobulin.

In the hybridoma method of generating monoclonal antibodies, a mouse or other appropriate host animal, such as a hamster, is immunized as hereinabove described to elicit lymphocytes that produce or are capable of producing antibodies that will specifically bind to the protein used for immunization (see generally, U.S. Pat. No. 5,914,112, which is incorporated herein by reference in its entirety.)

Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Human myeloma and mouse-human heteromyeloma cell lines also have been described for the production of human monoclonal antibodies (Kozbor, 1984, J. Immunol., 133:3001; Brodeur et al., Monoclonal Antibody Production Techniques and Applications, pp. 51-63 (Marcel Dekker, Inc., New York, 1987)). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immuno-absorbent assay(ELISA). The binding affinity of the monoclonal antibody can, for example, be determined by the Scatchard analysis of Munson et al., 1980, Anal. Biochem., 107:220.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Alternative to preparing monoclonal antibody-secreting hybridomas, a monoclonal antibody directed against a pathogen or pathogenic antigenic molecule polypeptide of the invention can be identified and isolated by screening a recombinant combinatorial immunoglobulin library (e.g., an antibody phage display library) with the antigen of interest. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

In addition, techniques developed for the production of “chimeric antibodies” (Morrison, et al., 1984, Proc. Natl. Acad. Sci., 81, 6851-6855; Neuberger, et al., 1984, Nature 312, 604-608; Takeda, et al., 1985, Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.)

Humanized antibodies are antibody molecules from non-human species having one or more complementarity determining regions (CDRs) from the non-human species and a framework region from a human immunoglobulin molecule. (see e.g., U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. Nos. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Natl. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

Complementarity determining region (CDR) grafting is another method of humanizing antibodies. It involves reshaping murine antibodies in order to transfer full antigen specificity and binding affinity to a human framework (Winter et al. U.S. Pat. No. 5,225,539). CDR-grafted antibodies have been successfully constructed against various antigens, for example, antibodies against IL-2 receptor as described in Queen et al., 1989 (Proc. Natl. Acad. Sci. USA 86:10029); antibodies against cell surface receptors-CAMPATH as described in Riechmann et al. (1988, Nature, 332:323; antibodies against hepatitis B in Cole et al. (1991, Proc. Natl. Acad. Sci. USA 88:2869); as well as against viral antigens-respiratory syncitial virus in Tempest et al. (1991, Bio-Technology 9:267). CDR-grafted antibodies are generated in which the CDRs of the murine monoclonal antibody are grafted into a human antibody. Following grafting, most antibodies benefit from additional amino acid changes in the framework region to maintain affinity, presumably because framework residues are necessary to maintain CDR conformation, and some framework residues have been demonstrated to be part of the antigen binding site. However, in order to preserve the framework region so as not to introduce any antigenic site, the sequence is compared with established germline sequences followed by computer modeling.

Completely human antibodies are particularly desirable for therapeutic treatment of human patients. Such antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of an immunogen.

Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif. (see, for example, U.S. Pat. No. 5,985,615)) and Medarex, Inc. (Princeton, N.J.), can be engaged to provide human antibodies directed against a selected antigen using technology similar to that described above.

Completely human antibodies which recognize and bind a selected epitope can be generated using a technique referred to as “guided selection.” In this approach a selected non-human monoclonal antibody, e.g., a mouse antibody, is used to guide the selection of a completely human antibody recognizing the same epitope (Jespers et al. (1994) antigen Bio/technology 12:899-903).

A pre-existing antibody directed against a pathogen can be used to isolate additional antigens of the pathogen by standard techniques, such as affinity chromatography or immunoprecipitation for use as immunogens. Moreover, such an antibody can be used to detect the protein (e.g., in a cellular lysate or cell supernatant) in order to evaluate the abundance and pattern of expression of the pathogen. The antibodies can also be used diagnostically to monitor pathogen levels in tissue as part of a clinical testing procedure, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

Antibodies that are commercially available can be purchased and used to generate bispecific antibodies, e.g., from ATCC. In a preferred embodiment of the invention, the antibody is produced by a commercially available hybridoma cell line. In a more preferred embodiment, the hybridoma secretes a human antibody.

Antibodies obtained by any of the methods or any combination of the methods are then combined into a polyclonal population of antibodies, which can be used in the production of the polyconal population of bispecific molecules.

### 5.3.3. Production and Purification of Polyclonal Populations of Bispecific Molecules

The polyclonal population of bispecific molecules of the invention is prepared by chemical cross-linking a population of anti-CR1 portions and a polyclonal population of antigen recognition portions. Any standard chemical conjugation methods can be used in the present invention. Cross-linking agents, including but are not limited to, protein A, glutaraldehyde, carbodiimide, N-succinimidyl-S-acetyl-thioacetate (SATA), N-succinimidyl-3-(2-pyridyldithio)propionate (SPDP), and sulfosuccinimidyl 4-(N-maleimidomethyl) cyclohexane-1-carboxylate (sulfo-SMCC) can be used (see e.g., Paulus, 1985, Behring Ins. Mitt. No. 78: 118-132; Karpovsky et al. 1984, J. Exp. Med. 160:1686; Liu, Mass. et al., 1985, Proc. Natl. Acad. Sci. USA 82:8648; Brennan et al., 1985 Science 229:81-83; and Glennie et al., 1987, J. Immunol. 139: 2367-2375; Perez et al, 1985, Nature 316:354-356; and Titus et al, 1987, Journal of Immunology 139:3153-3158).

In a preferred embodiment, cross-linking agents N-succinimidyl S-acetylthioacetate (SATA) and sulfosuccinimidy 4-(N-maleimidomethyl)cyclohexane-1-carboxylate (sulfo-SMCC) (Pierce Chemical Co., Rockford, Ill.) are used to cross-link a population of anti-CR1 portions and a polyclonal population of antigen recognition portions (see e.g., Liu, Mass. et al., 1985, Proc. Natl. Acad. Sci. USA 82:8648). In another preferred embodiment, N-succinimidyl 3-(2-pyridyldithio)propionate (SPDP) is used to cross-link a population of anti-CR1 portions and a polyclonal population of antigen recognition portions (see e.g., Karpovsky et al. 1984, J. Exp. Med. 160:1686; Perez et al, 1985, Nature 316:354-356; and Titus et al, 1987, Journal of Immunology 139:3153-3158).

The population of bispecific molecules can be purified by methods known to one skilled in the art using molecular size or specific binding affinity or a combination thereof. In one embodiment, the bispecific molecules can be purified by ion exchange chromatography using columns suitable for isolation of the bispecific molecules of the invention including DEAE, Hydroxylapatite, Calcium Phosphate (see generally Current Protocols in Immunology, 1994, John Wiley & Sons, Inc., New York, N.Y.).

In another embodiment, the population of bispecific molecules are purified by three-step successive affinity chromatography (Corvalan and Smith, 1987, Cancer Immunol. Immunother., 24:127-132): the first column is made of protein A bound to a solid matrix, wherein the Fc portion of the antibody binds protein A, and wherein the antibodies bind the column; followed by a second column that utilizes C3b-like receptor bound to a solid matrix which assays for C3b-like receptor binding via the anti-CR1 mAb portion of the bispecific molecule; and followed by a third column that utilizes specific binding of an antigenic molecule of interest which binds the antigen recognition portion of the bispecific molecule.

The population of bispecific molecules can also be purified by a combination of size exclusion HPLC and affinity chromatography. In one embodiment, the appropriate fraction eluted from size exclusion HPLC is further purified using a column containing an antigenic molecule specific to the antigen recognition portion of the bispecific molecule.

The population of bispecific molecules may also be isolated by isoelectric focusing of antibodies.

The population of bispecific molecules can be characterized by various methods known in the art. In one embodiment, the bispecific molecules can be characterized by SDS-PAGE and Western blot. The molecular weight of the bispecific molecule is determined by SDS-PAGE. The bispecificity of the molecules in the appropriate band is then determined by Western blots using both CR1 and the antigenic molecule of interest. Alternatively, the bispecifity of the molecules can be determined by solid-phase immunoassays, such as enzyme-linked immunosorbent assays (ELISA).

Synergistic polyclonal populations of bispecific molecules are produced by incorporating bispecific molecules that show synergistic activity in neutralization of a pathogen or a pathogenic antigenic molecule into the polyclonal population. In one embodiment, monoclonal bispecific molecules are produced using any methods known in the art, e.g., Himawan, U.S. Provisional Patent Application Ser. Nos. 60/199,903, 60/244,811, and 60/244,812; Taylor, U.S. Pat. Nos. 5,470,570; 5,487,890; and 5,879,679 or obtained from public and/or private sources. The activities in neutralization of a pathogen or pathogenic antigenic molecule by such monoclonal bispecific molecules are determined and compared with the activities of the polyclonal population using standard methods known in the art (see, e.g., Laal et al., 1994, J. Virol. 68:4001-4008; Burkly et al., J. Virol. 69:4267-4273; Vijh-Warrier et al., 1996, J. Virol. 70:4466-4473; Mascola et al., 1997, J. Virol. 71:7198-7206; Schaefer et al., 2000, Infection and Immunity 68:2608-2616). Monoclonal bispecific molecules that show synergistic activities when combined with the polyclonal population are incorporated into the polyclonal bispecific population to produce a synergistic population of bispecific molecules. In another embodiment, monoclonal antibodies that show synergistic activities when combined with a polyclonal preparation of antibodies are first combined with the polyclonal preparation to form a synergistic polyclonal population of antibodies. The synergistic polyclonal population of antibodies is then cross-linked with a population of anti-CR1 portions to produce a synergistic polyclonal population of bispecific molecules.

### 5.4. Uses of Polyclonal Population of Bispecific Molecules

The bispecific molecules of the present invention are useful in treating or preventing in a patient a disease or disorder associated with the presence of a pathogenic antigenic molecule. A patient can be a human or a mammalian nonhuman animal, including but are not limited to farm animals and pets. The pathogenic antigenic molecule can be any substance that is present in the circulation that is potentially injurious to or undesirable in the subject to be treated, including but not limited to proteins or drugs or toxins, autoantibodies or autoantigens, or a molecule of any infectious agent or its products. A pathogenic antigenic molecule is any molecule containing an antigenic determinant (or otherwise capable of being bound by a binding domain) that is or is part of a substance (e.g., a pathogen) that is the cause of a disease or disorder or any other undesirable condition.

Circulating pathogenic antigenic molecules cleared by the fixed tissue phagocytes include any antigenic moiety that is harmful to the subject. Examples of harmful pathogenic antigenic molecules include any pathogenic antigen associated with a parasite, fungus, protozoa, bacteria, or virus. Furthermore, circulating pathogenic antigenic molecules may also include toxins, immune complexes, autoantibodies, drugs, an overdose of a substance, such as a barbiturate, or anything that is present in the circulation and is undesirable or detrimental to the health of the host mammal. Failure of the immune system to effectively remove the pathogenic antigenic molecules from the mammalian circulation can lead to traumatic and hypovolemic shock (Altura and Hershey, 1968, Am. J. Physiol. 215:1414-9).

Moreover, non-pathogenic antigens, for example transplantation antigens, are mistakenly perceived to be harmful to the host and are attacked by the host immune system as if they were pathogenic antigenic molecules. The present invention further provides an embodiment for treating transplantation rejection comprising administering to a subject an effective amount of a bispecific antibody that will bind and remove immune cells or factors involved in transplantation rejection, e.g., transplantation antigen specific antibodies.

### 5.4.1. Using Polyclonal Populations of Bispecific Molecules for Removing Pathogens

The polyclonal population of bispecific molecules can be used to treat or prevent diseases by removing the disease-causing pathogens from the circulatory system. The polyclonal population of bispecific molecules is particularly useful in the clearance of highly mutable pathogens by targeting multiple epitopes on a pathogen. The polyclonal population is also particularly useful in the clearance of pathogens consisting multiple variants. The polyclonal population is further particularly useful in the clearance of heterogeneous mixtures of pathogens.

### 5.4.2. Target Antigenic Molecules

The present invention provides methods of treating or preventing a disease or disorder associated with the presence of a pathogenic antigenic molecule. The pathogenic antigenic molecule can be any substance that is present in the circulation that is potentially injurious to or undesirable in the subject to be treated, including but are not limited to proteins or drugs or toxins, autoantibodies or autoantigens, or a molecule of any infectious agent or its products. A pathogenic antigenic molecule is any molecule containing an antigenic determinant (or otherwise capable of being bound by a binding domain) that is or is part of a substance (e.g., a pathogen) that is the cause of a disease or disorder or any other undesirable condition.

Circulating pathogenic antigenic molecules cleared by the fixed tissue phagocytes include any antigenic moiety that is harmful to the subject. Examples of harmful pathogenic antigenic molecules include any pathogenic antigen associated with a parasite, fungus, protozoa, bacteria, or virus. Furthermore, circulating pathogenic antigenic molecules may also include toxins, immune complexes, autoantibodies, drugs, an overdose of a substance, such as a barbiturate, or anything that is present in the circulation and is undesirable or detrimental to the health of the host mammal. Failure of the immune system to effectively remove the pathogenic antigenic molecules from the mammalian circulation can lead to traumatic and hypovolemic shock (Altura and Hershey, 1968, Am. J. Physiol. 215:1414-9).

Moreover, non-pathogenic antigens, for example transplantation antigens, are mistakenly perceived to be harmful to the host and are attacked by the host immune system as if they were pathogenic antigenic molecules. The present invention further provides an embodiment for treating transplantation rejection comprising administering to a subject an effective amount of a bispecific antibody that will bind and remove immune cells or factors involved in transplantation rejection, e.g., transplantation antigen specific antibodies.

### 5.4.2.1. Naturally Occurring Antigens and Autoimmune Antigens

In one embodiment, the pathogenic antigenic molecule to be cleared from the circulation includes naturally occurring antigens and autoimmune antigens. These antigens include but are not limited to autoantibodies or naturally occurring molecules associated with autoimmune diseases.

Many different naturally occurring antigens and autoantibodies can be cleared from the circulation of a primate by using the polyclonal population of bispecific antibodies of the present invention. In a non-limiting example, IgE (immunoglobulin E) antibodies are cleared from the circulation by the bispecific antibodies of the invention. More specifically, the polyclonal population of bispecific antibodies comprises a first antigen recognition portion that is specific to a C3b-like receptor and a plurality of different second antigen recognition portions that is specific to IgE molecules. This population of bispecific antibodies can be used to decrease circulating IgE antibodies thereby reducing or inhibiting allergic reactions such as asthma.

In another example, certain humans with hemophilia have been shown to be deficient in factor VIII. Recombinant factor VIII replacement treats this hemophilia. However, eventually some patients develop antibodies against factor VIII, thus interfering with the therapy. The polyclonal population of bispecific antibodies of the present invention prepared with anti-anti-factor VIII antibodies provides a therapeutic solution for this problem. In particular, a polyclonal population of bispecific antibodies with specificity of a first antigen recognition portion to C3b-like receptor and specificities of a plurality second antigen recognition portions to anti-factor VIII autoantibodies would be therapeutically useful in clearing the autoantibodies from the circulation, thus, ameliorating the disease.

Further examples of autoantibodies which can be cleared by the polyclonal population of bispecific antibodies of the present invention include, but are not limited to, autoantibodies to the following antigens: the muscle acetylcholine receptor (the antibodies are associated with the disease myasthenia gravis); cardiolipin (associated with the disease lupus); platelet associated proteins (associated with the disease idiopathic thrombocytopenic purpurea); the multiple antigens associated with Sjogren's Syndrome; the antigens implicated in the case of tissue transplantation autoimmune reactions; the antigens found on heart muscle (associated with the disease autoimmune myocarditis); the antigens associated with immune complex mediated kidney disease; the dsDNA and ssDNA antigens (associated with lupus nephritis); desmogleins and desmoplakins (associated with pemphigus and pemphigoid); or any other antigen which is characterized and is associated with disease pathogenesis.

When the above populations of bispecific antibodies are injected into the circulation of a human or non-human primate, the bispecific antibodies will bind to red blood cells via the human or primate C3b receptor recognition site, at a high percentage and in agreement with the number of C3b-like receptor sites on red blood cells. The bispecific antibodies will simultaneously associate with the autoantibody through the antigen, which is bound to the moiety that binds the C3b-like receptor. The red blood cells which have the bispecific antibody/autoantibody complex on their surface then facilitate the neutralization and clearance from the circulation of the bound pathogenic autoantibody.

In the present invention, the polyclonal population of bispecific antibodies facilitates pathogenic antigen or autoantibody binding to hematopoietic cells expressing a C3b-like receptor on their surface and subsequently clear the pathogenic antigen or autoantibody from the circulation, without also clearing the hematopoietic cells.

### 5.4.2.2. Infectious Diseases

In specific embodiments, infectious diseases are treated or prevented by administration of a polyclonal population of bispecific molecules that bind both one or more antigens of one or more infectious disease agent and a C3b-like receptor. Thus, in such an embodiment, the pathogen or pathogens are antigens of one or more infectious disease agent. The polyclonal population of bispecific antibodies of the invention is particularly useful in clearing highly mutable and/or highly heterogeneous infectious disease agents.

Such antigen can be but is not limited to: influenza virus hemagglutinin (Genbank accession no. J02132; Air, 1981, Proc. Natl. Acad. Sci. USA 78:7639-7643; Newton et al., 1983, Virology 128:495-501), human respiratory syncytial virus G glycoprotein (Genbank accession no. Z33429; Garcia et al., 1994, J. Virol.; Collins et al., 1984, Proc. Natl. Acad. Sci. USA 81:7683), core protein, matrix protein or other protein of Dengue virus (Genbank accession no. M19197; Hahn et al., 1988, Virology 162:167-180), measles virus hemagglutinin (Genbank accession no. M81899; Rota et al., 1992, Virology 188:135-142), herpes simplex virus type 2 glycoprotein gB (Genbank accession no. M14923; Bzik et al., 1986, Virology 155:322-333), poliovirus I VP1 (Emini et al., 1983, Nature 304:699), envelope glycoproteins of HIV I (Putney et al., 1986, Science 234:1392-1395), hepatitis B surface antigen (Itoh et al., 1986, Nature 308:19; Neurath et al., 1986, Vaccine 4:34), diphtheria toxin (Audibert et al., 1981, Nature 289:543), streptococcus 24M epitope (Beachey, 1985, Adv. Exp. Med. Biol. 185:193), gonococcal pilin (Rothbard and Schoolnik, 1985, Adv. Exp. Med. Biol. 185:247), pseudorabies virus g50 (gpD), pseudorabies virus II (gpB), pseudorabies virus gIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydodysenteriae protective antigen, bovine viral diarrhea glycoprotein 55, Newcastle disease virus hemagglutinin-neuraminidase, swine flu hemagglutinin, swine flu neuraminidase, foot and mouth disease virus, hog colera virus, swine influenza virus, African swine fever virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein I), a glycoprotein of La Crosse virus (Gonzales-Scarano et al., 1982, Virology 120: 42), neonatal calf diarrhea virus (Matsuno and Inouye, 1983, Infection and Immunity 39:155), Venezuelan equine encephalomyelitis virus (Mathews and Roehrig, 1982, J. Immunol. 129:2763), punta toro virus (Dalrymple et al., 1981, Replication of Negative Strand Viruses, Bishop and Compans (eds.), Elsevier, N.Y., p. 167), murine leukemia virus (Steeves et al., 1974, J. Virol. 14:187), mouse mammary tumor virus (Massey and Schochetman, 1981, Virology 115:20), hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651-693; Tiollais et al., 1985, Nature 317:489-495), of equine influenza virus or equine herpesvirus (e.g., equine influenza virus type A/Alaska 91 neuraminidase, equine influenza virus type A/Miami 63 neuraminidase, equine influenza virus type A/Kentucky 81 neuraminidase equine herpesvirus type 1 glycoprotein B, and equine herpesvirus type 1 glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and the bovine parainfluenza virus type 3 hemagglutinin neuraminidase), bovine viral diarrhea virus glycoprotein 48 or glycoprotein 53.

Additional diseases or disorders that can be treated or prevented by the use of a polyclonal population of bispecific molecules of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, hantavirus, coxsachie virus, mumps virus, measles virus, rubella virus, polio virus, human immunodeficiency virus type I (HIV-I), and human immunodeficiency virus type II (HIV-II), any picornaviridae, enteroviruses, caliciviridae, any of the Norwalk group of viruses, togaviruses, such as Dengue virus, alphaviruses, flaviviruses, coronaviruses, rabies virus, Marburg viruses, ebola viruses, parainfluenza virus, orthomyxoviruses, bunyaviruses, arenaviruses, reoviruses, rotaviruses, orbiviruses, human T cell leukemia virus type I, human T cell leukemia virus type II., simian immunodeficiency virus, lentiviruses, polyomaviruses, parvoviruses, Epstein-Barr virus, human herpesvirus-6, cercopithecine herpes virus 1 (B virus), and poxviruses

Bacterial diseases or disorders that can be treated or prevented by the use of bispecific molecules of the present invention include, but are not limited to, those caused by Mycobacteria rickettsia, Mycoplasma, Neisseria spp. (e.g., Neisseria menigitidis and Neisseria gonorrhoeae), Legionella, Vibrio cholerae, Streptococci, such as Streptococcus pneumoniae, Corynebacteria diphtheriae, Clostridium tetani, Bordetella pertussis, Haemophilus spp. (e.g., influenzae), Chlamydia spp., enterotoxigenic Escherichia coli, and Bacillus anthracis (anthrax), etc.

Protozoal diseases or disorders that can be treated or prevented by the use of bispecific molecules of the present invention include, but are not limited to, plasmodia, eimeria, Leishmania, and trypanosoma.

### 5.4.2.3. Reptiles or Insects Poisoning

Envenomation by reptiles or insects often leads to the deposition of a mixture of toxic substances into the blood stream of the victim. The toxic substances in such a mixture are structurally heterogenous. The clinical symptom, i.e., poisoning, is a result of multiple blood-borne toxins. In one embodiment of the invention, such a mixture of toxic substances are targeted and cleared by the administration of an effective amount of a polyclonal population of bispecific molecules that bind both the toxic substances that cause the poisoning and a C3b-like receptor.

### 5.4.2.4. Additional Pathogenic Antigenic Molecules

In one embodiment, the pathogenic antigenic molecule or pathogenic antigenic molecules to be cleared from the circulation by the methods and compositions of the present invention encompass any serum drug, including but not limited to barbiturates, tricyclic antidepressants, and Digitalis.

In another embodiment, the pathogenic antigenic molecule or pathogenic antigenic molecules to be cleared includes any serum antigen that is present as an overdose and can result in temporary or permanent impairment or harm to the subject. This embodiment particularly relates to drug overdoses.

In another embodiment, the pathogenic antigenic molecule or pathogenic antigenic molecules to be cleared from the circulation include naturally occurring substances. Examples of naturally occurring pathogenic antigenic molecules that could be removed by the methods and compositions of the present invention include but are not limited to low density lipoproteins, interleukins or other immune modulating chemicals and hormones.

### 5.4.3. Dose of Bispecific Antibodies

The dose can be determined by a physician upon conducting routine experiments. Prior to administration to humans, the efficacy is preferably shown in animal models. Any animal model for a circulatory disease known in the art can be used.

More particularly, the dose of the polyclonal population of bispecific antibodies can be determined based on the hematopoietic cell concentration and the number of C3b-like receptor epitope sites bound by the anti-C3b-like receptor monoclonal antibodies per hematopoietic cell. If the bispecific antibodies are added in excess, a fraction of the bispecific antibodies will not bind to hematopoietic cells, and will inhibit the binding of pathogenic antigens to the hematopoietic cell. The reason is that when the free bispecific antibodies are in solution, they will compete for available pathogenic antigens with bispecific antibodies bound to hematopoietic cells. Thus, the bispecific antibody-mediated binding of the pathogenic antigens to hematopoietic cells follows a bell-shaped curve when binding is examined as a function of the concentration of the input bispecific antibodies concentration.

Viremia may result in up to 108-109 viral particles/ml of blood (HIV is 10⁶/ml; (Ho, 1997, J. Clin. Invest. 99:2565-2567)); the dose of therapeutic bispecific antibodies should preferably be, at a minimum, approximately 10 times the antigen number in the blood.

In general, for antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration are often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

As defined herein, a therapeutically effective amount of bispecific antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but are not limited to the severity of the disease or disorder, previous treatments, the general health and/or age of the subject, and other diseases present. Moreover, treatment of a subject with a therapeutically effective amount of a polyclonal population of bispecific antibodies can include a single treatment or, preferably, can include a series of treatments. In a preferred example, a subject is treated with a polyclonal population of bispecific antibodies in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a polyclonal population of bispecific antibodies, used for treatment may increase or decrease over the course of a particular treatment. Changes in dosage may result and become apparent from the results of diagnostic assays as described herein.

It is understood that appropriate doses of polyclonal populations of bispecific antibodies depend upon a number of factors within the ken of the ordinarily skilled physician, veterinarian, or researcher. The dose(s) of the bispecific antibody will vary, for example, depending upon the identity, size, and condition of the subject or sample being treated, further depending upon the route by which the composition is to be administered, if applicable, and the effect which the practitioner desires the polyclonal population of bispecific antibodies to have upon a pathogenic antigenic molecules or autoantibodies.

It is also understood that appropriate doses of polyclonal populations of bispecific antibodies depend upon the potency of the bispecific antibodies with respect to the antigen or antigens to be cleared. Such appropriate doses may be determined using the assays described herein. When one or more of these bispecific antibodies is to be administered to an animal (e.g., a human) in order to clear an antigen, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the bispecific antibodies employed, the age, body weight, general health, gender, and diet of the subject, the time of administration, the route of administration, the rate of excretion, any drug combination, and the concentration of antigen to be cleared.

### 5.4.4. Pharmaceutical Formulation and Administration

The polyclonal population of bispecific antibodies of the Invention can be incorporated into pharmaceutical compositions suitable for administration. Such compositions typically comprise a polyclonal population of bispecific antibodies and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the bispecific antibodies, use thereof in the compositions is contemplated.

A pharmaceutical composition of the invention is formulated to be compatible with its intended route of administration. The preferred route of administration is intravenous. Other examples of routes of administration include parenteral, intradermal, subcutaneous, transdermal (topical), and transmucosal. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that the viscosity is low and the bispecific antibodies is injectable. It must be stable under the conditions of manufacture and storage and must be preserved against the contaminating action of microorganisms such as bacteria and fungi.

The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the a polyclonal population of bispecific antibodies in the required amount in an appropriate solvent with one or a combination of ingredients enumerated above, as required, followed by filtered sterilization. Generally, dispersions are prepared by incorporating the polyclonal population of bispecific antibodies into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In one embodiment, the polyclonal population of bispecific antibodies are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 which is incorporated herein by reference in its entirety.

It is advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of a polyclonal population of bispecific antibodies calculated to produce the desired therapeutic effect in association with the required pharmaceutical carrier. The specification for the dosage unit forms of the invention are dictated by and directly dependent on the unique characteristics of the polyclonal population of bispecific antibodies and the particular therapeutic effect to be achieved, and the limitations inherent in the art of compounding such a polyclonal population of bispecific antibodies for the treatment of individuals.

The pharmaceutical compositions can be included in a kit, in a container, pack, or dispenser together with instructions for administration.

In some embodiments of the invention, the polyclonal population of bispecific molecules can be administered in conjunction with one or more monoclonal bispecific molecules. In a preferred embodiment, the polyclonal population of bispecific molecules is administered together with one or more monoclonal bispecific molecules that show complementary effects to the polyclonal population such that the combined therapy has enhanced effectiveness.

The polyclonal population of bispecific molecules can also be administered in conjunction with one or more monoclonal antibodies each comprising an antigen recognition portion. Such monoclonal antibodies increase the effectiveness of a therapy of the polyclonal population, e.g., by enhancing the binding affinity of one or more bispecific molecules in the polyclonal population. In a preferred embodiment, the polyclonal population of bispecific molecules is administered together with one or more monoclonal antibodies that have complementary effects to the polyclonal population such that the combined therapy has enhanced effectiveness.

### 5.4.5. Ex Vivo Preparation of the Bispecific Molecule

In an alternative embodiment, the bispecific molecules, such as a bispecific antibodies, is prebound to hematopoietic cells of the subject ex vivo, prior to administration. For example, hematopoietic cells are collected from the individual to be treated (or alternatively hematopoietic cells from a non-autologous donor of the compatible blood type are collected) and incubated with an appropriate dose of the therapeutic polyclonal population of bispecific antibodies for a sufficient time so as to allow the antibody to bind the C3b-like receptor on the surface of the hematopoietic cells. The hematopoietic cell/bispecific antibodies mixture is then administered to the subject to be treated in an appropriate dose (see, for example, Taylor et al., U.S. Pat. No. 5,487,890).

The hematopoietic cells are preferably blood cells, most preferably red blood cells.

Accordingly, in a specific embodiment, the invention provides a method of treating a mammal having an undesirable condition associated with the presence of one or more pathogens or pathogenic antigenic molecules, comprising the step of administering a polyclonal population of hematopoietic cell/bispecific molecule complexes to the subject in a therapeutically effective amount, each member of the polyclonal population consisting essentially of a hematopoietic cell expressing a C3b-like receptor bound to one or more bispecific molecules, wherein each of said bispecific molecule consists of a first antigen recognition portion, e.g., a monoclonal antibody to CR1, chemically cross-linked to a second antigen recognition portion, e.g., a monoclonal antibody which binds the pathogen, and wherein the polyclonal population comprising members which binds to different recognition sites of the one or more pathogens or pathogenic antigenic molecules. The method alternatively comprises a method of treating a mammal having an undesirable condition associated with the presence of a pathogenic antigenic molecule comprising the steps of (a) contacting a polyclonal population of bispecific molecules having a plurality of bispecific molecules that recognize different recognition sites of one or more pathogens or pathogenic antigenic molecules with hematopoietic cells expressing a C3b-like receptor, to form a polyclonal population of hematopoietic cell/bispecific molecule complexes, wherein each bispecific molecule consists of a first antigen recognition portion, e.g., a monoclonal antibody to CR1, chemically cross-linked to a second antigen recognition portion, e.g., a monoclonal antibody which binds the pathogenic antigenic molecule; and (b) administering the polyclonal population of hematopoietic cell/bispecific molecule complexes to the mammal in a therapeutically effective amount.

The invention also provides a method of making a polyclonal population of hematopoietic cell/bispecific olecule complexes comprising contacting a polyclonal population of bispecific molecules with hematopoietic cells that express a C3b-like receptor under conditions conducive to binding, such that complexes form, said complexes consisting essentially of a hematopoietic cell bound to one or more bispecific molecules, wherein each said bispecific molecule comprises a first binding domain that binds the C3b-like receptor on the hematopoietic cells cross-linked to a different second binding domain that binds a pathogen.

Taylor et al. (U.S. Pat. No. 5,879,679, hereinafter “the '679 patent”) have demonstrated in some instances that the system saturates because the concentration of autoantibodies (or other pathogenic antigen) in the plasma is so high that even at the optimum input of bispecific antibodies, not all of the autoantibodies can be bound to the hematopoietic cells under standard conditions. For example, for a very high titer of autoantibody sera, a fraction of the autoantibody is not bound to the hematopoietic cells due to its high concentration.

However, saturation can be solved by using combinations of bispecific antibodies which contain monoclonal antibodies that bind to different sites on a C3b-like receptor. For example, the monoclonal antibodies 7G9 and 1B4 bind to separate and non-competing sites on the primate C3b receptor. Therefore, a “cocktail” containing a mixture of two bispecific antibodies, each made with a different monoclonal antibody to the C3b-like receptor, may give rise to greater binding of antibodies to red blood cells. The bispecific antibodies of the present invention can also be used in combination with certain fluids used for intravenous infusions.

In yet another embodiment, the polyclonal population of bispecific molecules, such as a polyclonal population of bispecific antibodies, is prebound to red blood cells in vitro as described above, using a blend of at least two different first mAbs which bind to distinct and non-overlapping recognition sites on the C3b-like receptor. By using at least two non-overlapping first mAbs for binding to the C3b-like receptor, the number of bispecific antibody-antigen complexes that can bind to a single red blood cell is increased. Thus, by allowing more than one bispecific antibody to bind to a single C3b-like receptor, antigen clearance is enhanced, particularly in cases where the antigen is in very high concentrations (see for example the '679 patent, column 6, lines 41-64).

### 5.5. Kits

The invention provides kits containing the polyclonal population of bispecific molecules of the invention, or the polyclonal population of bispecific molecules of the invention and one or more monoclonal bispecific molecules that are synergistic in neutralization of a pathogen or pathogenic antigenic molecule with the polyclonal population, in one or more containers. The invention also provides kits containing the polyclonal population of bispecific molecules of the invention and one or more monoclonal antibodies each comprising an antigen recognition portion and is synergistic with the polyclonal population, in one or more containers. Kits containing the pharmaceutical compositions of the invention are also provided.

### 6. EXAMPLE

### Preparation and Uses of Anti-HIV-1 Polyclonal Population of Bispecific Molecules

The following example describes the production and uses of a polyclonal population of bispecific molecules comprising a plurality of bispecific molecules each having a first antigen recognition portion that binds a CR1 receptor cross-linked to a second antigen recognition portion that binds an HIV-1 virus. This example is a preferred embodiment of the invention.

HIV-1 is a highly mutable virus that during the course of HIV-1 infection, the antibodies generated in an infected individual do not provide permanent protective effect due in part to the rapid emergence of neutralization escape variants (Thali et al., 1992, J. Acquired Immune Deficiency Syndromes 5:591-599). Polyclonal preparations of antibodies, such as hyperimmune anti-HIV IgG preparations obtained from the plasma of multiple infected donors, have been shown to offer certain advantages in the recognition and neutralization of a broad range of HIV isolates (Cummins et al., 1991, Blood 77:1111-1117; Lambert et al., 1997, J. Infectious Diseases 175:283-291). Therefore, a polyclonal population of bispecific molecules comprising a plurality of different bispecific molecules each comprising a first antigen recognition portion that binds a CR1 receptor cross-linked to a different second antigen recognition portion that binds an HIV-1 virus is advantageous in providing patients with high therapeutic and/or prophylactic efficacy against HIV-1 infection.

Hybridoma cell line ATCC HB 8592 is obtained from the American Type Culture Collection (ATCC). Hybridomas are grown to log phase in Dulbecco's Modified Eagle's Medium (DMEM). The hybridoma cells are first washed in PBS. The cells are then resuspended in 1 ml buffer GTC (4M Guanidine-Isothiocyanate, 25 mM Sodium Citrate, 0.5% Sarcoyl, 0.1M β-mercaptoethanol). 0.1 ml sodium acetate (3M, pH 5.2), 0.5 ml phenol, and 0.2 ml choloroform are then added to the cell suspension. The cell suspension is then centrifuged at 10,000×g for 15 minutes. Supernatant is precipitated using 1 volume of isopropanol and is centrifuged at 10,000×g for 15 minutes. The pellet is washed in 70% EtOH and allowed air dry before is resuspended in 100 μl DEPC-treated water. Anti-CR1 mAbs are then recovered from the supernatants.

Preparation of purified polycolonal anti-HIV-1 immunoglobulin (HIVI\acute{G}) is obtained from various sources, e.g., NABI (Boca Raton, Fla.), Chromaprobe (Mountain View, Calif.), BioHeme (Salt Lake City, Utah), or National Institute of Health (www.aidsreagent.com). HIVIG is purified from multiple HIV-1-positive donors selected from geographically diverse regions of the United States. The product is a 50-mg/ml solution containing 98% monomeric immunogobulin G.

The preparation of purified polyclonal anti-HIV-1 immunoglobulin is cross-linked with the preparation of anti-CR1 mAbs using N-succinimidyl-3-(2-pyridyldithio)propionate (SPDP) (Pharmacia Fine Chemicals, Piscataway, N.J.) according to the manufacturer's protocol. The cross-linked bispecific molecules are fractionated by gel filtration on a 2.6×90 cm Ultrogel ACA34 column to remove non-cross-linked molecules.

Bi-specificity of the population of cross-linked bispecific molecules is demonstrated according to the method described in Taylor, U.S. Pat. Nos. 5,470,570; 5,487,890; and 5,879,679.

The polyclonal population of bispecific molecules is used as therapeutic agent for removing HIV-1 viral particles from the bloodstream of infected patients. A 5 mg dose of the polyclonal population (preferably in liquid form) is administered parenterally to an infected adult patient of normal size. The level of HIV-1 viral particles in the patient's bloodstream is monitored using any standard viral load assay known in the art, e.g., AMPLICOR HIV-1 MONITOR from Roche Diagnostics (Indianapolis, Ind.). The administration of the polyclonal population reduces the blood HIV-1 level by several order of magnitude within the first hour of administration.

The polyclonal population of bispecific molecules is also used prophylactically. In prophylactic uses, the polyclonal population is administered once monthly to people of high risk to HIV infection, including but not limited to health care personnel and sexual partners of infected patients. As a prophylaxis, the polyclonal population of bispecific molecules prevents HIV-1 viral particles from reaching a sustained and appreciable level.

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claims is:

1. A polyclonal population of bispecific molecules comprising a plurality of different bispecific molecules, each bispecific molecule in said plurality comprising a first antigen recognition portion that binds a C3b-like receptor cross-linked to a different second antigen recognition portion that binds a pathogenic antigenic molecule.

2. The polyclonal population of bispecific molecules of claim 1, wherein said first antigen recognition portion comprises an anti-CR1 monoclonal antibody.

3. The polyclonal population of bispecific molecules of claim 1, wherein said plurality of bispecific molecules comprises different bispecific molecules comprising different first antigen recognition portions.

4. The polyclonal population of bispecific molecules of claim 1, wherein said second antigen recognition portion comprises a monoclonal antibody.

5. The polyclonal population of bispecific molecules of claim 1, wherein said plurality of bispecific molecules comprises bispecific molecules comprising different second antigen recognition portions having different antigen recognition specificities directed to different epitopes on a pathogen.

6. The polyclonal population of bispecific molecules of claim 1, wherein said plurality of bispecific molecules comprises bispecific molecules comprising different second antigen recognition portions having different antigen recognition specificities directed to different variants of a pathogen.

7. The polyclonal population of bispecific molecules of claim 1, wherein said plurality of bispecific molecules comprises bispecific molecules comprising different second antigen recognition portions having different antigen recognition specificities directed to different pathogens.

8. The polyclonal population of bispecific molecules of claim 5, comprising bispecific molecules that are synergistic in neutralization of said pathogen.

9. The polyclonal population of bispecific molecules of any one of claims 1-8, wherein said plurality of bispecific molecules constitutes at least 10% of said population.

10. The polyclonal population of bispecific molecules of any one of claims 1-8, wherein said plurality of bispecific molecules constitutes at least 20% of said population.

11. The polyclonal population of bispecific molecules of any one of claims 1-8, wherein said plurality of bispecific molecules constitutes at least 50% of said population.

12. The polyclonal population of bispecific molecules of any one of claims 1-8, wherein said plurality of bispecific molecules constitutes at least 80% of said population.

13. The polyclonal population of bispecific molecules of any one of claims 1-8, wherein said plurality of bispecific molecules constitutes at least 90% of said population.

14. The polyclonal population of bispecific molecules of any one of claims 1-8, wherein no single bispecific molecule has a proportion exceeding 95% of said plurality.

15. The polyclonal population of bispecific molecules of any one of claims 1-8, wherein no single bispecific molecule has a proportion exceeding 80% of said plurality.

16. The polyclonal population of bispecific molecules of any one of claims 1-8, wherein no single bispecific molecule has a proportion exceeding 60% of said plurality.

17. The polyclonal population of bispecific molecules of any one of claims 1-8, wherein no single bispecific molecule has a proportion exceeding 50% of said plurality.

18. The polyclonal population of bispecific molecules of any one of claims 1-8, wherein said plurality of different bispecific molecules comprises at least 2 different bispecific molecules having different second antigen recognition portions.

19. The polyclonal population of bispecific molecules of any one of claims 1-8, wherein said plurality of different bispecific molecules comprises at least 10 different bispecific molecules having different second antigen recognition portions.

20. The polyclonal population of bispecific molecules of any one of claims 1-8, wherein said plurality of different bispecific molecules comprises at least 100 different bispecific molecules having different second antigen recognition portions.

21. The polyclonal population of bispecific molecules of any one of claims 1-8, wherein said population is purified.

22. The polyclonal population of bispecific molecules of any one of claims 1-8, wherein said second antigen recognition portions are from a hyperimmune serum.

23. A polyclonal population of bispecific molecules comprising a plurality of bispecific molecules that are produced by cross-linking a plurality of first antigen recognition portions that bind C3b-like receptors and a plurality of different second antigen recognition portions that bind pathogenic antigenic molecules.

24. The polyclonal population of bispecific molecules of claim 23, wherein said plurality of different second antigen recognition portions is from a hyperimmune serum.

25. A method for producing a polyclonal population of bispecific molecules, comprising cross-linking a plurality of first antigen recognition portions that bind C3b-like receptors and a plurality of different second antigen recognition portions that bind pathogenic antigenic molecules.

26. A method for producing a polyclonal population of bispecific molecules, comprising:
(a) producing a plurality of first antigen recognition portions that bind a C3b-like receptor;
(b) producing a plurality of different second antigen recognition portions that bind one or more pathogenic antigenic molecules; and
(c) cross-linking said plurality of first antigen recognition portions and said plurality of different second antigen recognition portions, to produce said polyclonal population of bispecific molecules.

27. The method of claim 25 or 26, wherein said plurality of different second antigen recognition portions is from a hyperimmune serum.

28. The method of claim 25 or 26, wherein said plurality of different second antigen recognition potions is obtained from a phage display library.

29. The method of claim 28, wherein said plurality of second antigen recognition portions is produced by a method comprising expressing in a host a plurality of eukaryotic expression vectors each containing a nucleotide sequence encoding the heavy and light chain variable regions of a different immunoglobulin in a polyclonal library of immunoglobulin molecules, wherein said nucleotide sequences encoding the heavy and light chain variable regions comprise nucleotide sequences encoding the respective heavy and light chains linked head to head to form a bidirectional vector.

30. The method of claim 28, wherein said plurality of second antigen recognition portions is produced by a method comprising:
(a) selecting from a phage display library a plurality of phages that display antigen recognition polypeptides having different respective binding specificities to one or more antigenic molecules using affinity screening;
(b) obtaining a plurality of nucleic acids encoding said plurality of antigen recognition polypeptides, respectively; and
(c) expressing said plurality of nucleic acids in a host, to produce said polyclonal population of second antigen recognition portions.

31. The method of claim 25 or 26, wherein said cross-linking comprises using cross-linking agents N-succinimidyl S-acetylthioacetate and sulfosuccinimidy 4-(N-maleimidomethyl)cyclohexane-1-carboxylate.

32. The method of claim 25 or 26, wherein said cross-linking comprises using cross-linking agent N-succinimidyl 3-(2-pyridyldithio)propionate (SPDP).

33. A method for producing a synergistic polyclonal population of bispecific molecules comprising a plurality of different bispecific molecules, each bispecific molecule in said plurality comprising a first antigen recognition portion that binds a C3b-like receptor cross-linked to a different second antigen recognition portion that binds an epitope of the same pathogen or pathogenic antigenic molecule, said method comprising combining a polyclonal population of bispecific molecules with one or more monoclonal bispecific molecules that show synergistic activities in neutralization of said pathogen or pathogenic antigenic molecule when combined with said polyclonal population, to produce a synergistic polyclonal population of bispecific molecules.

34. A method for producing a synergistic polyclonal population of bispecific molecules, comprising:
(a) producing a plurality of first antigen recognition portions that bind C3b-like receptors;
(b) producing a plurality of different second antigen recognition portions that bind the same pathogen or pathogenic antigenic molecule;
(c) cross-linking said plurality of first antigen recognition portions and said plurality of different second antigen recognition portions to produce a polyclonal population of bispecific molecules;
(d) obtaining one or more monoclonal second antigen recognition portions that are synergistic in neutralization of said pathogen or pathogenic antigenic molecule;
(e) cross-linking said first antigen recognition portion and said one or more monoclonal second antigen recognition portions to produce one or more monoclonal bispecific molecules; and
(f) incorporating in said polyclonal population of bispecific molecules said one or more monoclonal bispecific molecules, to produce a synergistic polyclonal population of bispecific molecules.

35. A method of treating a mammal having a disease or disorder or undesirable condition associated with the presence of one or more pathogens or pathogenic antigenic molecules, comprising administering to said mammal a therapeutically effective dose of a polyclonal population of bispecific molecules comprising a plurality of different bispecific molecules, each bispecific molecule in said plurality comprising a first antigen recognition portion that binds a C3b-like receptor cross-linked to a different second antigen recognition portion that binds an epitope of said one or more pathogens or pathogenic antigenic molecules.

36. A method of preventing a disease or disorder or undesirable condition associated with the presence of one or more pathogens or pathogenic antigenic molecules in a mammal, comprising administering prior to the onset of said disease or disorder or undesirable condition, to said mammal a prophylactically effective dose of a polyclonal population of bispecific molecules comprising a plurality of different bispecific molecules, each bispecific molecule in said plurality comprising a first antigen recognition portion that binds a C3b-like receptor cross-linked to a different second antigen recognition portion that binds an epitope of said one or more pathogens or pathogenic antigenic molecule.

37. A method of treating a mammal having a disease or disorder or undesirable condition associated with the presence of one or more pathogens or pathogenic antigenic molecules, comprising administering to said mammal a therapeutically effective dose of a polyclonal population of bispecific molecules and one or more monoclonal bispecific molecules, said polyclonal population of bispecific molecules comprising a plurality of different bispecific molecules, each bispecific molecule in said plurality comprising a first antigen recognition portion that binds a C3b-like receptor cross-linked to a different second antigen recognition portion that binds a pathogenic antigenic molecule, said monoclonal bispecific molecules are synergistic in neutralization of said one or more pathogens or pathogenic antigenic molecules when combined with said polyclonal population.

38. The method of claim 35, 36 or 37, wherein said administering is intravenous.

39. The method of claim 35, 36 or 37, wherein said mammal is a human, and said C3b-like receptor is CR1.

40. The method of claim 35, 36 or 37, wherein said mammal is a non-human mammal.

41. The method of claim 35, 36 or 37, wherein said pathogens comprise an autoimmune antigen.

42. The method of claim 35, 36 or 37, wherein said pathogens comprise an infectious agent.

43. The method of claim 42, wherein said infectious agent is a virus.

44. The method of claim 43, wherein said virus is HIV-1 virus.

45. The method of claim 42, wherein said infectious agent is a bacterium.

46. The method of claim 42, wherein said infectious agent is a fungus.

47. The method of claim 42, wherein said infectious agent is a parasite.

48. The method of claim 47, wherein said parasite is a protozoan.

49. The method of claim 35, 36 or 37, wherein said pathogens comprise a poisonous compound.

50. The method of claim 49, wherein said poisonous compound is a poisonous compound contained in a venom.

51. The method of claim 50, wherein said venom is reptile venom.

52. The method of claim 51, wherein said venom is snake venom.

53. The method of claim 51, wherein said venom is insect venom.

54. The method of claim 35, 36 or 37, wherein said plurality of bispecific molecules comprises bispecific molecules comprising different second antigen recognition portions having different antigen recognition specificities directed to different epitopes of the same pathogen or pathogenic antigenic molecule.

55. The method of claim 35, 36 or 37, wherein said plurality of bispecific molecules comprises bispecific molecules comprising different second antigen recognition portions having different antigen recognition specificities directed to different variants of a pathogen.

56. The method of claim 35, 36 or 37, wherein said plurality of bispecific molecules comprises bispecific molecules comprising different second antigen recognition portions having different antigen recognition specificities directed to different pathogens.

57. The method of claim 35, 36 or 37, wherein said plurality of different bispecific molecules comprising at least 2 different bispecific molecules having different second antigen recognition portions.

58. The method of claim 35, 36 or 37, wherein said plurality of different bispecific molecules comprises at least 10 different bispecific molecules having different second antigen recognition portions.

59. The method of claim 35, 36 or 37, wherein said plurality of different bispecific molecules comprises at least 100 different bispecific molecules having different second antigen recognition portions.

60. The method of claim 35, 36 or 37, wherein said first antigen recognition portion comprises an anti-CR1 monoclonal antibody.

61. The method of claim 35, 36 or 37, wherein said plurality of bispecific molecules comprises different bispecific molecules comprising different first antigen recognition portions.

62. The method of claim 35, 36 or 37, wherein said second antigen recognition portion comprises a monoclonal antibody.

63. The method of claim 35, 36 or 37, wherein said plurality of bispecific molecules constitutes at least 10% of said population.

64. The method of claim 35, 36 or 37, wherein said plurality of bispecific molecules constitutes at least 20% of said population.

65. The method of claim 35, 36 or 37, wherein said plurality of bispecific molecules constitutes at least 50% of said population.

66. The method of claim 35, 36 or 37, wherein said plurality of bispecific molecules constitutes at least 80% of said population.

67. The method of claim 35, 36 or 37, wherein said plurality of bispecific molecules constitutes at least 90% of said population.

68. The method of claim 35, 36 or 37, wherein said plurality of bispecific molecules constitutes at least 95% of said population.

69. The method of claim 35, 36 or 37, wherein no single bispecific molecule has a proportion exceeding 80% of said plurality.

70. The method of claim 35, 36 or 37, wherein no single bispecific molecule has a proportion exceeding 60% of said plurality.

71. The method of claim 35, 36 or 37, wherein no single bispecific molecule has a proportion exceeding 50% of said plurality.

72. A kit comprising in a container a polyclonal population of bispecific molecules comprising a plurality of different bispecific molecules, each bispecific molecule in said plurality comprising a first antigen recognition portion that binds a C3b-like receptor cross-linked to a different second antigen recognition portion that binds a pathogenic antigenic molecule.

73. A kit comprising in two or more containers a) a polyclonal population of bispecific molecules comprising a plurality of different bispecific molecules, each bispecific molecule in said plurality comprising a first antigen recognition portion that binds a C3b-like receptor cross-linked to a different second antigen recognition portion that binds a pathogenic antigenic molecule; and b) one or more monoclonal bispecific molecules each comprising a first antigen recognition portion that binds a C3b-like receptor and a second antigen recognition portion that binds said pathogenic antigenic molecule, said one or more monoclonal bispecific molecules are synergistic with said plurality in neutralization of said pathogenic antigenic molecule.

74. A kit comprising in two or more containers a) a polyclonal population of bispecific molecules comprising a plurality of different bispecific molecules, each bispecific molecule in said plurality comprising a first antigen recognition portion that binds a C3b-like receptor cross-linked to a different second antigen recognition portion that binds a pathogenic antigenic molecule; and b) one or more monoclonal antibodies each comprising an antigen recognition portion that binds said pathogenic antigenic molecule, said one or more monoclonal antibodies are synergistic with said plurality in neutralization of said pathogenic antigenic molecules.

75. The kit of claim 72, 73 or 74, wherein said plurality of different bispecific molecules comprises at least 2 different bispecific molecules having different second antigen recognition portions.

76. The kit of claim 72, 73 or 74, wherein said plurality of different bispecific molecules comprises at least 10 different bispecific molecules having different second antigen recognition portions.

77. The kit of claim 72, 73 or 74, wherein said plurality of different bispecific molecules comprises at least 100 different bispecific molecules having different second antigen recognition portions.

78. The kit of claim 72, 73 or 74, wherein said first antigen recognition portion in said plurality comprises an anti-CR1 monoclonal antibody.

79. The kit of claim 72, 73 or 74, wherein said plurality of bispecific molecules comprises different bispecific molecules comprising different first antigen recognition portions.

80. The kit of claim 72, 73 or 74, wherein said second antigen recognition portion in said plurality comprises a monoclonal antibody.

81. The kit of claim 80, wherein said plurality of bispecific molecules comprises bispecific molecules comprising different second antigen recognition portions having different antigen recognition specificities directed to different epitopes of the same pathogen.

82. The kit of claim 72, 73 or 74, wherein said plurality of bispecific molecules constitutes at least 10% of said population.

83. The kit of claim 72, 73 or 74, wherein said plurality of bispecific molecules constitutes at least 20% of said population.

84. The kit of claim 72, 73 or 74, wherein said plurality of bispecific molecules constitutes at least 50% of said population.

85. The kit of claim 72, 73 or 74, wherein said plurality of bispecific molecules constitutes at least 80% of said population.

86. The kit of claim 72, 73 or 74, wherein said plurality of bispecific molecules constitutes at least 90% of said population.

87. The kit of claim 72, 73 or 74, wherein said plurality of bispecific molecules constitutes at least 95% of said population.

88. The kit of claim 72, 73 or 74, wherein no single bispecific molecule has a proportion exceeding 80% of said plurality.

89. The kit of claim 72, 73 or 74, wherein no single bispecific molecule has a proportion exceeding 60% of said plurality.

90. The kit of claim 72, 73 or 74, wherein no single bispecific molecule has a proportion exceeding 50% of said plurality.

91. A population of modified hematopoietic cells that consists essentially of a population of hematopoietic cells each bound to one or more bispecific molecules, wherein each of said bispecific molecules comprises a first antigen recognition portion that binds a C3b-like receptor cross-linked to a different second antigen recognition portion that binds a pathogenic antigenic molecule, wherein said bispecific molecules bound to said population of modified hematopoietic cells forms a population of bispecific molecules comprising different second antigen recognition portions.

92. A polyclonal population of bispecific molecules comprising a plurality of different bispecific molecules, each bispecific molecule in said plurality comprising a first antigen recognition portion that binds a C3b-like receptor cross-linked to a different immunoglobulin molecule that binds a pathogen or pathogenic antigenic molecule.

93. The polyclonal population of bispecific molecules of claim 92, wherein said first antigen recognition portion comprises an anti-CR1 monoclonal antibody.

94. The polyclonal population of bispecific molecules of claim 92, wherein said plurality of bispecific molecules comprises different bispecific molecules comprising different first antigen recognition portions.

95. The polyclonal population of bispecific molecules of claim 92, wherein said different immunoglobulin molecules have different antigen recognition specificities directed to different epitopes of the same pathogen or pathogenic antigenic molecule.

96. The polyclonal population of bispecific molecules of claim 92, wherein said different immunoglobulin molecules have different antigen recognition specificities directed to different variants of a pathogen or pathogenic antigenic olecule.

97. The polyclonal population of bispecific molecules of claim 92, wherein said different immunoglobulin molecules have different antigen recognition specificities directed to different pathogens or pathogenic antigenic molecules.

98. The polyclonal population of bispecific molecules of claim 92, comprising bispecific molecules that are synergistic in neutralization of said pathogen or pathogenic molecule.

99. The polyclonal population of bispecific molecules of any one of claim 92-98, wherein said plurality of bispecific molecules constitutes at least 10% of said population.

100. The polyclonal population of bispecific molecules of any one of claims 92-98, wherein said plurality of bispecific molecules constitutes at least 20% of said population.

101. The polyclonal population of bispecific molecules of any one of claims 92-98, wherein said plurality of bispecific molecules constitutes at least 50% of said population.

102. The polyclonal population of bispecific molecules of any one of claims 92-98, wherein said plurality of bispecific molecules constitutes at least 80% of said population.

103. The polyclonal population of bispecific molecules of any one of claims 92-98, wherein said plurality of bispecific molecules constitutes at least 90% of said population.

104. The polyclonal population of bispecific molecules of any one of claims 92-98, wherein no single bispecific molecule has a proportion exceeding 95% of said plurality.

105. The polyclonal population of bispecific molecules of any one of claims 92-98, wherein no single bispecific molecule has a proportion exceeding 80% of said plurality.

106. The polyclonal population of bispecific molecules of any one of claims 92-98, wherein no single bispecific molecule has a proportion exceeding 60% of said plurality.

107. The polyclonal population of bispecific molecules of any one of claims 92-98, wherein no single bispecific molecule has a proportion exceeding 50% of said plurality.

108. The polyclonal population of bispecific molecules of any one of claims 92-98, wherein said plurality of different bispecific molecules comprises at least 2 different bispecific molecules.

109. The polyclonal population of bispecific molecules of any one of claims 92-98, wherein said plurality of different bispecific molecules comprises at least 10 different bispecific molecules.

110. The polyclonal population of bispecific molecules of any one of claims 92-98, wherein said plurality of different bispecific molecules comprises at least 100 different bispecific molecules.

111. The polyclonal population of bispecific molecules of any one of claims 92-98, wherein said population is purified.

112. The polyclonal population of bispecific molecules of any one of claims 92-98, wherein said bispecific molecules in said plurality comprise immunoglobulin molecules obtained from the same hyperimmune serum.
